(19) United States
(12) Patent Application Publication
(10) Pub. No.: US 2005/0255493 A1
(43) Pub. Date: Nov. 17, 2005

(54) Molecular targeting of the IGF-1 receptor

(76) Inventors: Valentine Moya Macaulay, Oxford (GB); Muhammad Sohail, Oxford (GB)

Correspondence Address: FOLEY & LARDNER LLP, 111 HUNTINGTON AVENUE, 26TH FLOOR, BOSTON, MA, 02199-7610, US

(21) Appl. No.: 10/996,951

(22) Filed: Nov. 24, 2004

Related U.S. Application Data
(63) Continuation of application No. PCT/GB03/02306, filed on May 28, 2003.

(30) Foreign Application Priority Data
May 28, 2002 (GB) 0212303.2

(51) Int. Cl. C12Q 1/68; C07H 21/02; A61K 48/00

(52) U.S. Cl. 435/6; 514/44; 536/23.1

(57) ABSTRACT

siRNA and antisense reagents capable of blocking expression of the IGF-1 receptor.

### FIELD OF THE INVENTION

The invention is concerned with RNAi and antisense reagents capable of blocking expression of the IGF-1 receptor.

### BACKGROUND TO THE INVENTION

The insulin-like growth factors-I and -II (IGFs-I and -II) and the type I IGF receptor (IGF1R) play a critical role in the establishment and maintenance of the transformed phenotype. High plasma IGF-1 levels have been shown to confer increased risk of prostate, colon and premenopausal breast cancer (Pollak 2000). IGF1R activation leads to growth, tumorigenesis, apoptosis protection and tumour cell motility (Macaulay 1992; Baserga 1997). IGF1R and its principal docking molecule insulin receptor substrate-1 can influence cell-cell interactions by modulating interaction between components of adherens junctions, including cadherin and beta-catenin (Playford et al 2000; Reiss et al 2000). Compared with normal tissues, the IGF1R is frequently overexpressed by tumours including colorectal cancer, melanoma and prostate cancer (Hellawell et al., Cancer Res. Vol. 62, 2942-50, 2002). Furthermore, IGF1R overexpression is associated with clinical radioresistance in breast cancer (Turner et al 1997). Tumour growth can be inhibited by blocking the expression or function of the IGF1R, strategies that also enhance the sensitivity of tumour cells to killing by cytotoxic drugs (Resnicoff et al, 1994; Liu et al 1998; Chernicky et al 2000, Sun et al 2001).

The present inventors have used antisense RNA to downregulate the IGF1R in mouse melanoma cells. This led to enhancement of radiosensitivity, reduced radiation-induced p53 accumulation and serine 18 phosphorylation, and radioresistant DNA synthesis (Macaulay et al., 2001). These changes were reminiscent of the cellular phenotype of the human genetic disorder ataxia telangiectasia (A-T), caused by mutations in the ATM gene. The ATM protein functions in the initiation of cell cycle checkpoints and DNA repair pathways after double strand breaks (Shiloh 2001). It was observed that antisense-IGF1R-transfected melanoma cells had lower ATM protein levels than controls, and failed to activate the ATM kinase after irradiation. This indicates that IGF1R signalling can modulate the expression and function of ATM, suggesting a key role for the IGF axis in the cellular response to DNA damage (Macaulay et al., 2001).

Clearly the IGF1R mediates many aspects of the malignant phenotype, and represents a highly promising therapeutic target. Thus far it has not proved possible to synthesise small molecule inhibitors with sufficient potency and specificity for the IGF1R versus other tyrosine kinases, particularly the insulin receptor. Molecular approaches are required for sequence-specific targeting of the IGF1R.

U.S. Pat. Nos. 5,643,788 and 6,340,674 relate to methods of inhibiting the proliferation and causing the differentiation of cells with a single-stranded antisense oligonucleotide having a sequence complementary to a region of the IGF-1 receptor mRNA. Two IGF1R antisense oligonucleotides are specifically described. The first is an oligodeoxynucleotide complementary to codons −29 to −24 of the signal sequence of the IGF-1 receptor, and the second is a ribodeoxynucleotide sequence complementary to codons 1 to 309 of the IGF-1 receptor.

Chernicky et al. (Cancer Gene Therapy, Vol 7, No. 3, 2000, pp 384-395) described the effects of treatment of human breast cancer cells with single-stranded antisense RNA to IGF1R. Treatment with an antisense RNA transcribed from a 637-bp cDNA fragment from nucleotide position 42 to exon 1 to nucleotide position 738 in exon 3 of the IGF1R cDNA (GenBank M24599) was shown to inhibit cell growth, suppress tumorigenesis, alter metastatic potential and prolong survival in vivo in this system.

Wraight et al. (Nature Biotechnology, Vol 18, pp521-526) demonstrated reversal of epidermal hyperplasia in a mouse model of psoriasis using IGF1R receptor antisense oligonucleotides. Computer modelling was used to select regions of the IGF1R transcript that lack internal duplex and hairpin structures. Over 80 15-mer antisense oligonucleotides corresponding to different regions of the IGF1R mRNA were synthesised and tested. Certain of these were shown to produce significant reduction in IGF1R mRNA expression, but others did not have any significant effect on mRNA expression.

The present inventors have now demonstrated that effective reduction of IGF1R expression can be achieved by RNA interference (RNAi) using a short double-stranded RNA. The inventors have further shown that RNAi can result in significantly more effective reduction in IGF1R expression as compared to antisense oligonucleotides of equivalent sequence. Thus, the invention provides an alternative method for down-regulating IGF1R expression.

### DESCRIPTION OF THE INVENTION

In a first aspect the invention provides an siRNA reagent comprising a double-stranded RNA sequence of up to 30 consecutive nucleotides of the human IGF-1 receptor mRNA sequence.

The siRNA reagents provided by the invention are useful as reagents mediating down-regulation of IGF1R1 expression by RNA interference. As aforesaid, the inventors have demonstrated by experiment that RNAi with short duplex RNAs can result in significantly more effective reduction in IGF1R expression as compared to antisense oligonucleotides of equivalent sequence.

The term “siRNA reagent” refers to a nucleic acid molecule that is capable of down-regulating expression of a target gene (i.e. IGF1R) by RNA interference. The characteristics of siRNA reagents are generally known in the art. siRNA reagents generally comprise a region of double-stranded RNA, although as discussed below one or more bases in the double-stranded RNA may be replaced with DNA bases. The double-stranded RNA may be flanked by short single-stranded overhangs, as described below.

The double-stranded RNA sequence incorporated into the siRNA reagent preferably comprises at least 15, and more preferably between 18 and 30 consecutive nucleotides of the human IGF-1 receptor mRNA sequence.

Preferably the double-stranded RNA will comprise up to 30 nucleotides of the human IGF-1 receptor mRNA sequence between position 537 and position 685. This region has been shown by the present inventors using RNase mapping to be accessible to RNAse-mediated cleavage.

Preferred double-stranded RNAs include those comprising between 18 and 30 consecutive nucleotides of the human IGF-1 receptor mRNA sequence between position 612 and position 661, preferably between position 612 and position 641 or between position 630 and position 661.

Further preferred double-stranded RNAs include those comprising between 18 and 30 consecutive nucleotides of the human IGF-1 receptor mRNA sequence immediately downstream of nucleotide number 636 or immediately downstream of nucleotide number 609.

Further preferred double-stranded RNAs include dsRNA homologous to a sequence nearer to the translation start site region of the IGF1R mRNA. These include dsRNAs comprising between 18 and 30 consecutive nucleotides of the human IGF-1 receptor mRNA sequence, immediately downstream of nucleotide number 166, or immediately upstream of nucleotide 186 and dsRNAs comprising between 18 and 30 consecutive nucleotides of the human IGF-1 receptor mRNA sequence including nucleotide number 176.

Most preferably the double-stranded RNA will be of identical sequence to an antisense oligonucleotide which hybridises to IGF1R mRNA with a relative intensity of at least 0.01 in a hybridisation buffer consisting of 1M NaCl, 10 mM Tris-HCl pH 7.4, 1 mM EDTA, 0.01% SDS (w/v) at a temperature of 37° C. Even more preferably the double-stranded RNA will be of identical sequence to an antisense oligonucleotide which hybridises to IGF1R mRNA with a relative intensity at least 0.28 in a hybridisation buffer consisting of 1M NaCl, 10 mM Tris-HCl pH 7.4, 1 mM EDTA, 0.01% SDS (w/v) at a temperature of 37° C.

Hybridisation between antisense oligonucleotides and IGF1R mRNA is most conveniently measured using a scanning oligonucleotide array, as described in the accompanying examples. Scanning arrays comprise sets of oligonucleotides of varying length complementary to a defined region of a selected mRNA target and enable parallel measurement of the binding of all oligonucleotides in the array to the target mRNA. For any given oligonucleotide a value of relative hybridisation intensity may be calculated relative to the oligonucleotide within the array which hybridises most strongly (i.e. with greatest intensity) under the selected hybridisation conditions by simply dividing hybridisation intensity of the chosen oligonucleotide by hybridisation intensity for the oligonucleotide within the array which hybridises most strongly. This calculation is illustrated in the accompanying examples for an IGF1R array.

The present inventors have shown not only that hybridisation intensity on an array is predictive of activity in cells for an antisense oligonucleotide (ASO), but also that hybridisation intensity on an array for a given ASO is predictive of the activity of a corresponding RNAi duplex of (substantially) identical sequence to the ASO. It was surprising to observe that sequences identified using the scanning array as being useful as antisense oligonucleotides may also mediate gene silencing by RNA interference, because the mechanisms of action of antisense oligonucleotides and RNA interference are different (see review by Brantl, S. Antisense-RNA regulation and RNA interference, Biochem Biophys Acta. Vol. 1575(1-3), 15-25, 2002).

The inventors have observed that siRNAs incorporating double-stranded RNAs of (substantially) identical sequence to antisense oligonucleotides which hybridise to IGF1R mRNA with a relative hybridisation intensity at least 0.01 in a hybridisation buffer consisting of 1M NaCl, 10 mM Tris-HCl pH 7.4, 1 mM EDTA, 0.01% SDS (w/v) at a temperature of 37° C. are effective in causing a reduction in IGF1R expression by RNAi when transfected into cancer cell lines.

The inventors have also shown that relative hybridisation intensity of a corresponding ASO to IGF1R mRNA is predictive of the activity of a double-stranded RNA in causing a reduction of IGF1R expression by RNAi. Thus, double-stranded RNAs corresponding in sequence to ASOs having a relative hybridisation intensity of significantly greater than 0.1 cause a more profound reduction in IGF1R expression. Double-stranded RNAs corresponding in sequence to ASOs having a relative hybridisation intensity of 0.28 or greater are particularly effective.

The ability of an siRNA reagent to down-regulate IGF1R expression in cell lines is also shown to be predictive of biological effects. For example, dsRNAs which cause significant reduction in IGF1R expression when transfected into cell lines have been demonstrated to block IGF-1 signalling to Akt. Akt phosphorylation is the major anti-apoptosis pathway downstream of the IGF1R. Cells transfected with dsRNAs which cause significant reduction in IGF1R expression in cell lines also showed reduced survival in vitro and reduced growth rate in vivo when introduced into mice.

When referring to the correspondence between an antisense oligonucleotide and a double-stranded RNA reagent (also referred to herein as an RNAi reagent, RNAi duplex or siRNA) the term “identical sequence” is not intended to be interpreted literally as requiring 100% sequence identity. The sequence of the dsRNA may differ slightly from the ASO sequence. For example the length of the dsRNA may be longer or shorter by several nt to optimise performance of the dsRNA. Most preferably, however, the double-stranded RNA region of the siRNA reagent will share at least 95%, and more preferably 100%, sequence identity with the antisense oligonucleotide (ASO) identified by array-hybridisation over the region of homology with the ASO.

The RNA duplex will preferably be less than 30 bp in length, since duplexes of greater than 30 bp may induce non-specific interferon-mediated effects when introduced into cells in vivo, and will preferably be at least 15 bp in length, more preferably at least 18 bp in length. RNA duplexes of 20-27 bp in length, or 20-24 bp in length, are particularly suitable as RNAi reagents. If the dsRNA is made longer than a corresponding ASO (e.g. an ASO identified on the basis of hybridisation to a scanning array) then the extra sequence may correspond to the “native” sequence of the mRNA. The dsRNA may contain one or more substitute bases in order to optimise performance in RNAi. As illustrated in the accompanying examples, substitution of even a single nucleotide may have a profound effect on activity of the RNAi duplex. It will be apparent to the skilled reader, following the teaching of the accompanying Examples, how to vary each of the bases of the dsRNA in turn and test the activity of the resulting siRNAs (e.g. in a suitable in vitro test system) in order to optimise the performance of a given siRNA. The dsRNA may further contain DNA bases, non-natural bases or non-natural backbone linkages, for example to enhance stability in vivo or enhance resistance to degradation by nucleases.

The dsRNA may also be flanked by single-stranded overhangs at one or both ends of the duplex. In a particularly preferred embodiment the dsRNA may contain 3′ overhanging nucleotides, preferably two 3′ overhanging thymidines (dTdT) or uridines (UU). 3′ TT or UU overhangs may be included in the RNAi duplex if the sequence of the target gene immediately upstream of the sequence included in double-stranded part of the RNAi duplex is AA. This allows the TT or UU overhang in the RNAi duplex to hybridise to the target gene. It is to be noted that sequences ASO2, ASO4 and ASO6 identified on the basis of array screening have AA immediately upstream, thus it is convenient to make RNAi equivalents to these sequences including 3′ overhangs. Although a 3′ TT or UU overhang may also be included at the other end of the RNAi duplex it is not essential for the target sequence downstream of the sequence included in double-stranded part of the RNAi duplex to have AA.

In a further embodiment rather than being formed of two separate RNA strands annealed together, the dsRNA may have a foldback stem-loop or hairpin structure, wherein the two strands of the dsRNA are covalently linked. RNAs having this structure are typical if the dsRNA is synthesised by expression in vivo or by in vitro transcription. The precise nature and sequence of the “loop” linking the two RNA strands is generally not material to the invention, except that it should not impair the ability of the double-stranded part of the molecule to mediate RNAi.

The double-stranded RNA will preferably comprise 20-27, or 20-24, consecutive nucleotides of the human IGF1R mRNA sequence, since duplexes of this length are particularly effective in RNAi.

siRNA reagents which are RNA/DNA chimeras are also contemplated. These chimeras include, for example, the siRNA reagents comprising a double-stranded RNA with 3′ overhangs of DNA bases (e.g. dTdT), as discussed above, and also siRNA reagents comprising a double-stranded “RNA” in which one or more of the RNA bases, or even an entire strand, are replaced with DNA bases.

The most preferred siRNA reagents include those having the following sequences, however these are given by way of example only and are not intended to be limiting to the invention:

| | |
|---|---|
| 5′-GCCGAUGUGUGAGAAGACCTT-3′ | (SEQ ID NO:23) |
| 3′-TTCGGCUACACACUCUUCUGG-5′ | |
| 5′-GCCGAUGUGUGAGAAGTT-3′ | (SEQ ID NO:24) |
| 3′-TTCGGCUACACACUCUUC-5′ | |
| 5′-GCCGAUGUGUGAGAAGACCACCTT-3′ | (SEQ ID NO: 25) |
| 3′-TTCGGCUACACACUCUUCUGGUGG-5′ | |
| 5′-GCCGAUGUGUGAGAAGACCACCAUCTT-3′ | (SEQ ID NO: 26) |
| 3′-TTCGGCUACACACUCUUCUGGUGGUAG-5′ | |
| 5′-CUACAUUGUGGGGAAUAAGTT-3′ | (SEQ ID NO: 34) |
| 3′-TTGAUGUAACACCCCUUAUUC-5′ | |
| 5′-CAAUGAGUACAACUACCGCTT-3′ | (SEQ ID NO: 30) |
| 3′-TTGUUACUCAUGUUGAUGGCG-5′ | |
| 5′-GCCCAUGUGUGAGAAGACCTT-3′ | (SEQ ID NO: 29) |
| 3′-TTCGGGUACACACUCUUCUGG-5′ | |
| 5′-CUACAUUGUGGGGAACAAGTT-3′ | (SEQ ID NO: 36) |
| 3′-TTGAUGUAACACCCCUUGUUC-5′ | |
| 5′-CGACUAUCAGCAGCUGAAGTT-3′ | (SEQ ID NO: 37) |
| 3′-TTGCUGAUAGUCGUCGACUUC-5′ | |

In a further embodiment the invention provides an siRNA reagent comprising a double-stranded RNA which is of identical sequence to an antisense oligonucleotide which hybridises to IGF1R mRNA and which exhibits greater specificity of hybridisation to IGF1R mRNA than to insulin receptor mRNA in a hybridisation buffer consisting of 1M NaCl, 10 mM Tris-HCl pH 7.4, 1 mM EDTA, 0.01% SDS (w/v) at a temperature of 37° C.

The nucleic acid sequence coding for the insulin receptor shares 60% homology with IGF1R therefore a significant problem in the design of RNAi and antisense reagents capable of down-regulating IGF1R expression is to avoid sequences which also hybridise to insulin receptor (IR) mRNA, and therefore down-regulate IR expression. This problem can be avoided with the use of scanning oligonucleotide array technology, since it is possible to select ASO sequences which selectively hybridise to IGF1R mRNA rather than IR mRNA and then synthesise RNA duplex reagents corresponding to these ASO sequences for use as RNAi reagents. Selectivity for the chosen mRNA target may be as important as simple hybridisation intensity in the design of RNAi reagents which are effective in vivo in a complex biological system.

siRNAs according to the invention may be synthesised in vitro using chemical or enzymatic RNA synthesis techniques well known in the art. In one approach the two separate RNA strands may be synthesised separately and then annealed to form double-strands (see examples)

In a further embodiment, siRNAs may be synthesised by intracellular expression from a suitable expression vector. Thus, the invention further provides an expression vector encoding an siRNA according to the invention. A number of non-viral (e.g. plasmid) or viral expression vector systems for in vivo expression of short double-stranded RNAs for use as RNAi reagents (also referred to as small interfering RNAs, or siRNAs) are known in the art. Generally, siRNAs are expressed as stem-loops, which may be rapidly processed within the cell to produce the “free” siRNA (see review by Tuschl, Nature Biotechnology, Vol. 20(5), 446-448, 2002). Vector systems for expression of siRNAs are often based on RNA Pol III promoters, since these are particularly suited to accurate expression of very short RNA sequences. Suitable vector systems are described in Brummelkamp, T. R. et al., Science, Vol. 296, 550-553, 2002; Lee, N. S. et al., Nature Biotechnology, Vol. 20, 500-505, 2002; Miyagashi, M & Taira, K. Nature Biotechnology, Vol. 20, 497-500, 2002; Paul, C. P. et al., Nature Biotechnology, Vol. 20, 505-508, 2002, the contents of which are incorporated herein by reference.

siRNAs may be formulated into pharmaceutical compositions comprising a therapeutically effective amount of the nucleic acid in combination with any standard physiologically and/or pharmaceutically acceptable carriers known in the art. “Pharmaceutically acceptable” means a non-toxic material which does not interfere with the activity of the pharmaceutically active ingredients in the composition. “Physiologically acceptable” refers to a non-toxic material that is compatible with a biological system such as a cell, tissue or organism. Physiologically and pharmaceutically acceptable carriers may include diluents, fillers, salts, buffers, stabilizers, solubilizers etc.

For delivery into cells in vivo siRNAs may be formulated with a lipid-based carriers including, for example, oil-in water emulsions, micelles, and liposomes. Liposomes are the most preferred carriers, and there use is well known in the art. Liposomes are commercially available from Gibco BRL, for example, as LIPOFECTIN™ and OLIGOFECTAMINE™, which are formed of cationic lipids. Methods for making liposomes are well known in the art and have been described in many publications. Liposomes may be targeted to a particular tissue by coupling the liposome to a specific ligand, such as a monoclonal antibody, sugar, glycolipid or protein.

Liposomes may also be used to deliver vectors encoding double-stranded RNAs. In the field of human gene therapy it is well known to deliver expression vectors, such as plasmids, via nucleic acid-liposome complexes.

For use in human therapy, pharmaceutical compositions including the siRNAs of the invention will be administered to a patient in need of treatment in a “therapeutically acceptable amount”. A therapeutically acceptable amount is an amount of a pharmaceutical preparation that alone, or together with further doses, produces the desired response in the condition being treated. The precise amount of the composition administered will, however, generally be determined by a medical practitioner, based on the circumstances pertaining to the disorder to be treated, such as the severity of the symptoms, the composition to be administered, the age, weight, and response of the individual patient and the chosen route of administration. The term “treatment” may encompass prophylactic treatment aimed at preventing the appearance or lessening the severity of disease symptoms.

The siRNA reagents of the invention may be used in vivo to block expression of IGF1R and hence inhibit tumour growth in malignant conditions such as, for example, melanoma, glioblastoma, human lung cancers, rhabdomyosarcoma, osteosarcoma, mesothelioma breast cancer and prostate cancer. These conditions have previously been shown to be responsive to antisense strategies that target IGF1R mRNA (melanoma, Resnicoff, M. et al., Cancer Res. 1994, Vol. 54, 4848-4850); glioblastoma, Resnicoff, M. et al., Cancer Res. 1994, Vol. 54, 2218-2222); human lung cancers, (Lee, C-T. et al., Cancer Res. 1996, Vol. 56, 3038-3041); rhabdomyosarcoma, (Shapiro, D. N. et al., J Clin Invest. 1994, Vol. 94, 1235-1242); osteosarcoma, (Kapel, C. C. Cancer Res. 1994, Vol. 54, 2803-2807); mesothelioma (Pass, H. I. Cancer Res. 1996, Vol. 54, 4044-4048); breast cancer (Chernicky et al., Cancer Gene Ther. &: 384-95, 2000); prostate cancer (Pietrzkowski, A. Cancer Res. 1993, Vol. 63, 1102-1106, Burfiend, P. et al., PNAS. 1996, Vol. 93, 7263-7268)). Since RNAi has been shown to be generally more effective than antisense in blocking IGF1R expression it is expected that siRNA reagents will be more potent inhibitors of tumour growth than antisense.

Blocking of IGF1R expression may also provide a means to enhance sensitivity to conventional treatments, for example radiotherapy, particularly in radioresistant cancers such as malignant melanoma, since downregulation of IGF1R expression has been shown to be associated with enhanced radiosensitity and impaired activation of Atm kinase in melanoma cells (Macaulay et al., Oncogene. Vol. 20, 4029-4040, 2001). In fact, the present inventor has shown that siRNA-mediated gene silencing enhances tumour cell radiosensitivity in MDA-MB-231 cells and in prostate cancer (see accompanying examples). Blocking of IGF1R expression may also enhance chemosensitivity. The inventor has shown that antisense-mediated IGF1R downregulation can enhance chemosensitivity (see accompanying examples and Hellawell et al, BJU International 91: 271-7, 2003).

The siRNA reagents provided by the invention are also useful in the treatment of non-malignant conditions for which IGF-1 signalling has a role in disease pathogenesis. A specific example is epidermal hyperproliferation in psoriasis. Inhibition of IGF1R expression has been proposed as an effective way of treating epidermal hyperplasia in psoriasis (Wraight et al., Nature Biotechnology, Vol. 18, 521-526, 2000).

In addition to double-stranded RNAs for use as RNAi reagents, the invention also provides a number of antisense oligonucleotides which are effective antisense reagents, causing significant reduction in IGF1R expression.

Therefore, in accordance with a second aspect of the invention there is provided an antisense oligonucleotide comprising at least 15 nucleotides which hybridises to IGF1R mRNA with a relative intensity of greater than 0.28 in a hybridisation buffer consisting of 1M NaCl, 10 mM Tris-HCl pH 7.4, 1 mM EDTA, 0.01% SDS (w/v) at a temperature of 37° C.

Most preferably the antisense oligonucleotide will be complementary to a region of the human IGF-1 receptor mRNA sequence between position 537 and position 685, a region which has shown to be accessible to RNase-mediated cleavage on the basis of RNase mapping.

Within the region from position 537 to 685 the inventors have shown that antisense oligonucleotides which bind to IGF1R mRNA with a relative hybridisation intensity of at least 0.28 are particularly effective in mediating reduction of IGF1R expression. Relative hybridisation intensity may be calculated as described above.

Particularly preferred antisense oligonucleotides include those having the follow sequences, however these are given by way of example only and are not intended to be limiting to the invention:

| | |
|---|---|
| 5′-GGCTTCTCCTCCATGGTC | (SEQ ID NO:1) |
| 5′-CGGCTTCTCCTCCATGGTCC | (SEQ ID NO:2) |
| 5′-GGTCTTCTCACACATCGG | (SEQ ID NO:6) |
| 5′-TGGTCTTCTCACACATCGGC | (SEQ ID NO:7) |
| 5′-GCGGTAGTTGTACTCATTGT | (SEQ ID NO:13) |

In a further embodiment the invention provides an antisense oligonucleotide comprising at least 15 nucleotides which hybridises to IGF1R mRNA and which exhibits greater specificity of hybridisation to IGF1R mRNA than to insulin receptor mRNA in a hybridisation buffer consisting of 1M NaCl, 10 mM Tris-HCl pH 7.4, 1 mM EDTA, 0.01% SDS (w/v) at a temperature of 37° C.

As stated above, a significant problem in the design of RNAi and antisense reagents capable of down-regulating IGF1R expression is to avoid sequences which also hybridise to insulin receptor (IR) mRNA, and therefore down-regulate IR expression. This problem can be avoided with the use of scanning oligonucleotide array technology, since it is possible to select ASO sequences which selectively hybridise to IGF1R mRNA rather than IR mRNA at 37° C.

The antisense oligonucleotides provided by the invention are single-stranded oligonucleotides of typically 18-25 nucleotides in length and may be composed of DNA or RNA. They may incorporate non-natural bases, for example C5 propyne analogs, and/or non-natural backbone linkages such as, for example, phosphorothioates, morpholino oligonucleotides, methylphosphonate backbones, MEA phosphoramidates, DEED phosphoramidates etc, and other modifications, for example 3′ terminal capping, in order to increase stability and enhance resistance to endonucleases.

Antisense oligonucleotides may be synthesised using chemical synthesis techniques which are well known in the art.

Antisense oligonucleotides may be formulated into pharmaceutical compositions comprising a therapeutically effective amount of the antisense nucleic acid in combination with any standard physiologically and/or pharmaceutically acceptable carriers known in the art.

For delivery into cells in vivo antisense oligonucleotides may be formulated with lipid-based carriers including, for example, cationic lipid carriers and liposomes, as discussed above. Lewis, J. G. et al., PNAS, Vol. 93, 3176-3181, 1996 describe a cationic lipid formulation (GS 2888 cytofectin) which may be used to deliver antisense oligonucleotides into a range of cell types.

Other approaches to delivery of antisense oligonucleotides include core-shell nanospheres (Tondelli. L. et al., J Biomater Sci Polym Ed, 2001, Vol. 12, 1339-57), biodegradable microspheres such as, for example, poly(lactide-co-glycolide) microspheres (Hussain, M. et al., Int J Pharm, 2002, Vol. 234, 129-38), and peptide-based vectors (Morris, M. C. et al., Nucleic Acids Research, Vol. 25, 2730, 1997). These approaches might also be adapted for the delivery of dsRNA reagents for RNAi.

Antisense compositions according to the invention would be used to reduce or inhibit the expression of IGF1R in vivo and are of therapeutic benefit in the same disease indications listed for RNAi reagents.

In a further aspect the invention provides a method of preparing an siRNA reagent for use in gene silencing of a target gene by RNA interference, which method comprises:
(a) preparing a scanning array of antisense oligonucleotides spanning a region of a transcript of the human IGF1R gene;
(b) hybridising to the array labelled transcripts of the IGF1R gene;
(c) identifying an oligonucleotide within the array which hybridizes with the labelled transcripts; and
(d) preparing an siRNA reagent comprising a double-stranded RNA of identical sequence to the oligonucleotide identified in step (c).

In this method a scanning array corresponding to a region of the IGF1R gene is synthesised using the known techniques (see references given above and accompanying examples).

The scanning arrays are hybridised with a probe which is a labelled transcript of the IGF1R gene. The probe may be labelled with essentially any type of revealing label which permits visualisation and quantitation of the hybridisation intensity. Radiolabels are particularly preferred. Suitable labelled RNA probes may be conveniently synthesised using standard techniques known in the art (see accompanying examples).

Typically the hybridisation will be carried out at a temperature of 37° C. Hybridisation at 37° C. is particularly preferred, since oligonucleotides which hybridise at this temperature are more likely to be effective in vivo. The compositions of standard hybridisation buffers which are preferred for use with the arrays are given in the accompanying examples.

Oligonucleotides which hybridise to the array are identified and si RNAs (also referred to herein as RNAi reagents or double-stranded RNA reagents) having identical sequence are synthesised. The inventors have demonstrated by experiment that hybridisation to the scanning array is directly predictive of effectiveness in RNA interference. In this context “identical sequence” is not intended to be interpreted literally as requiring 100% sequence identity, the sequence of the dsRNA may vary, as discussed above. The dsRNA may also be prepared by synthetic means, as described above.

In a further embodiment, siRNA reagents may be synthesised by intracellular expression from a suitable expression vector. Thus, the invention further provides a method of preparing an expression vector capable of expressing an siRNA for use in gene silencing of IGF1R by RNA interference, which method comprises:
(a) preparing a scanning array of antisense oligonucleotides spanning a region of a transcript of IGF1R;
(b) hybridising to the array labelled transcripts of IGF1R;
(c) identifying an oligonucleotide within the array which hybridizes with the labelled transcripts; and
(d) preparing an expression vector capable of expressing an siRNA comprising a double-stranded RNA of identical sequence to the oligonucleotide identified in step (c).

SiRNAs or expression vectors identified using the methods of the invention may subsequently be formulated into reagents for in vivo use.

The invention will be further understood with reference to the following experimental examples, together with the accompanying Figures, in which:

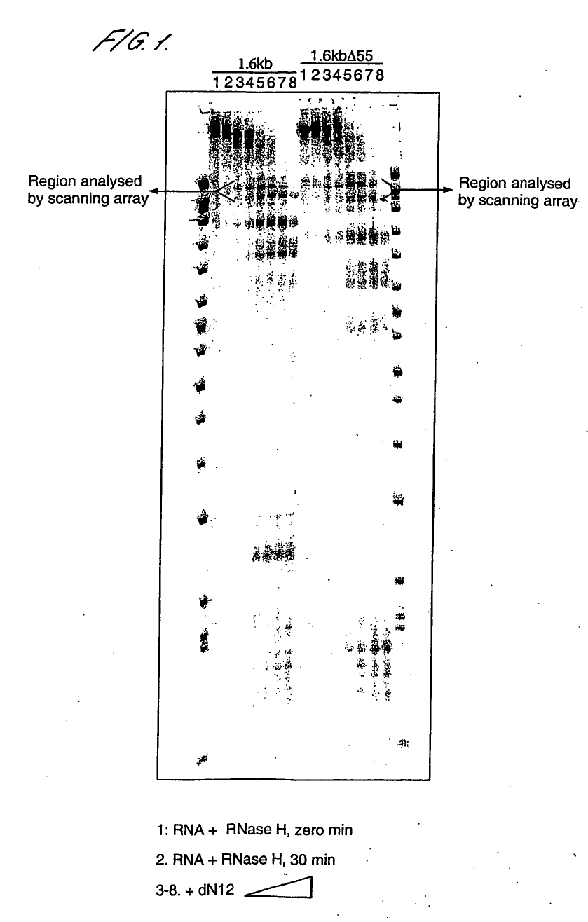

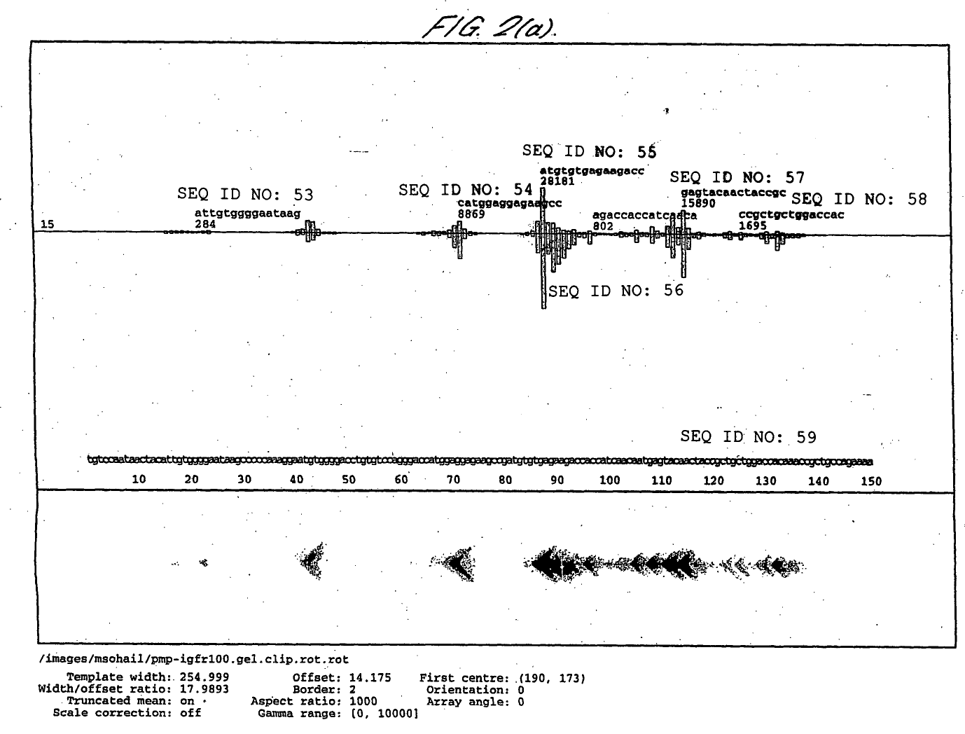

FIG. 1 is an autoradiograph showing gel analysis of the cleaved products of 5′-end labelled IGF1R mRNA with RNase H in the presence of a library of random 12mer oligonucleotides;

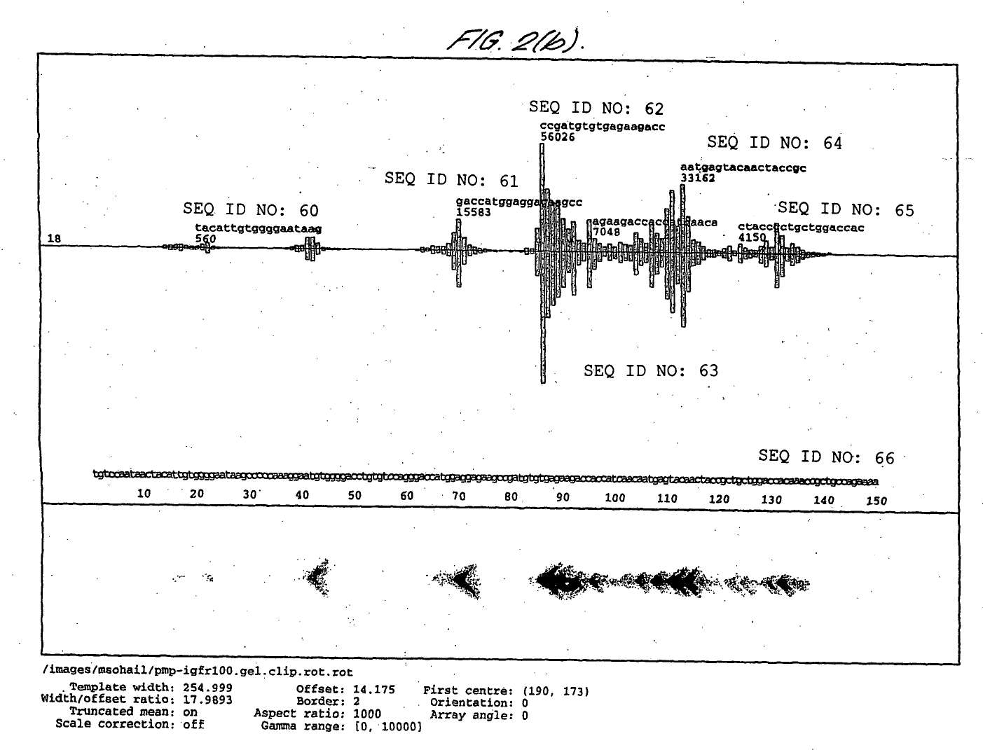

FIG. 2 shows representative plots of hybridisation intensity across the array for (a) 15mers, (b) 18mers and (c) 20 mers probed with labelled IGF1R mRNA. FIG. 2(c) further includes a comparison of the hybridization of IGF1R or IR mRNA to scanning array of IGF1R ASOs. Middle panel: hybridization to IGF1R transcript. Histogram (upper panel) represents quantification of binding of 20mer ASOs to IGF1R mRNA. Lower panel: hybridization to IR transcript. Numbered arrowheads: correspond to the ASO and siRNA sequences selected for further study. The 5′ end of the IGF1R sense sequence (Ullrich, A. et al., EMBO J, Vol 5, 2503-2512, 1986) targeted by ASO1 was base 596; ASO2, 612; ASO3, 622; ASO4, 637; ASO6, 546.

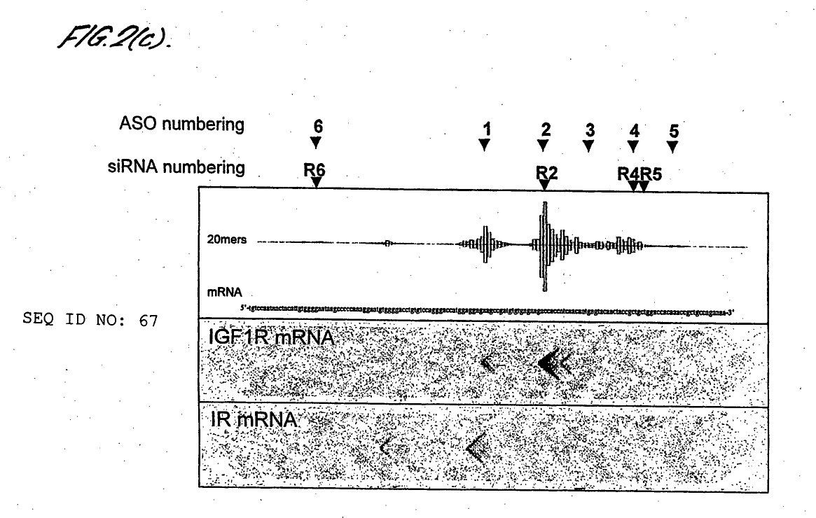

FIG. 3 illustrates the effect of ASOs and siRNA R2 on IGF1R levels in MDA-231 human breast cancer cells. The cells were transfected at 30-40% confluence with phosphorothioate ASOs complexed with the lipid Cytofectin (Glen GSV). After 48 hr the cells were lysed and equivalent amounts of soluble protein were separated by SDS-PAGE and immunoblotted for IGF1R and β-tubulin (loading control). The intensity of the autoradiographic bands was quantified by densitometry, and IGF1R levels were corrected for loading differences. The results are shown as % IGF1R level of that in cells transfected with the same concentration of an appropriate control. This was a scrambled control oligonucleotide for ASOs, and an inverted RNA duplex for siRNA R2.

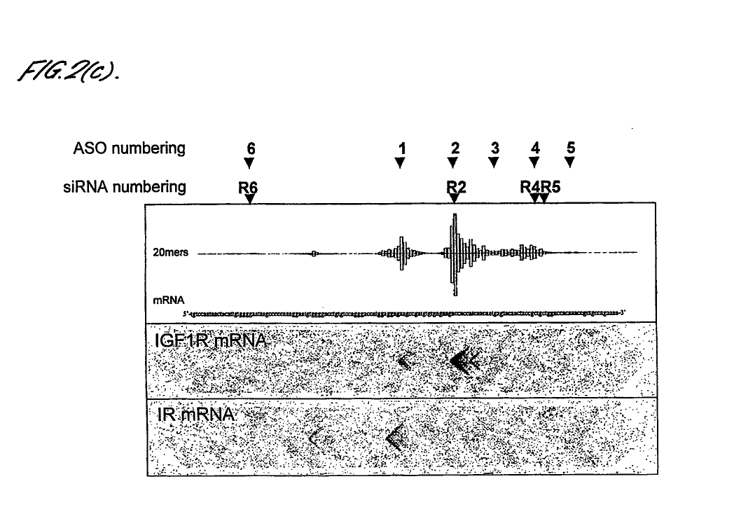

FIG. 4 illustrates the effect of ASOs and siRNA on IGF1R and IR levels in MDA-231 human breast cancer cells and ME melanoma cells. Cells were transfected using either Cytofectin (C) or Oligofectamine (O; Gibco BRL). After 48 hr the cells were lysed and IGF1R and IR levels were determined by immunoblotting.
a) MDA-231 breast cancer cells transfected with ASO 2 (or scrambled control, Scr) or siRNA R2 or inverted RNA duplex control (InvRNA) at 20 or 200 nM.
b) MDA-231 cells were transfected with Oligofectamine and siRNA R2 or Inverted control (InvRNA) at 0.1-10 nM.
c) ME melanoma cells were transfected with Oligofectamine and siRNA R2 or Inv control duplex at 5 -500 nM.

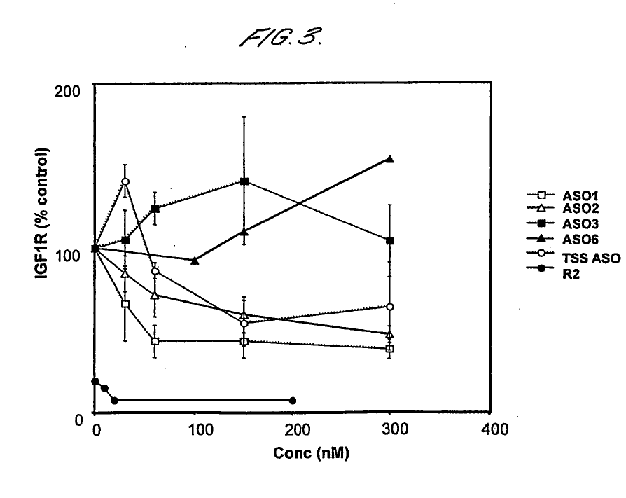

FIG. 5 illustrates the effects of various RNAi duplexes in MDA-231 breast cancer cells. MDA-231 cells were transfected with oligofectamine and 21mer RNA duplexes at 0.5, 5 and 50 nm. After 48 hr IGF1R expression was analysed by immunoblotting (panel (a)). Panel (b) is a graphical illustration, results are presented as % IGF1R level of that in cells transfected with the same concentration of an inverted control RNAi

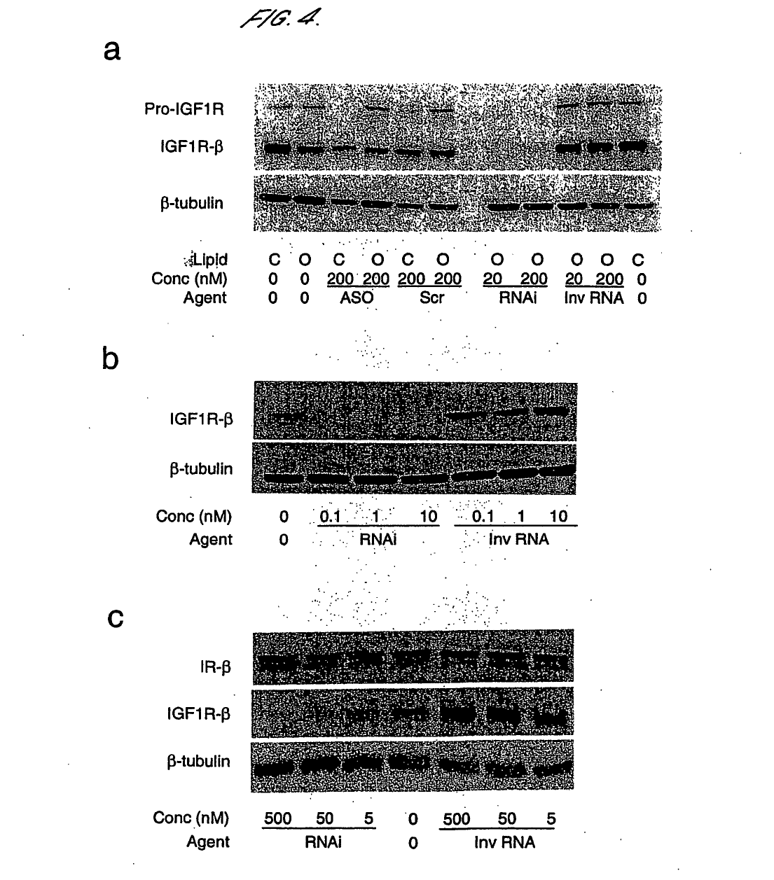

FIG. 6 illustrates the activity of RNAi duplexes of 18, 21, 24 and 27 nt in MDA-231 breast cancer cells. Panel (a) is an immunoblot showing the effect of various RNAis on IGF1R expression at 0.5 and 5 nM; panel (b) is a graphical illustration showing the effect of RNAis of varying length, results are presented as % IGF1R level of that in cells transfected with the same concentration of an Inv2 control RNAi.

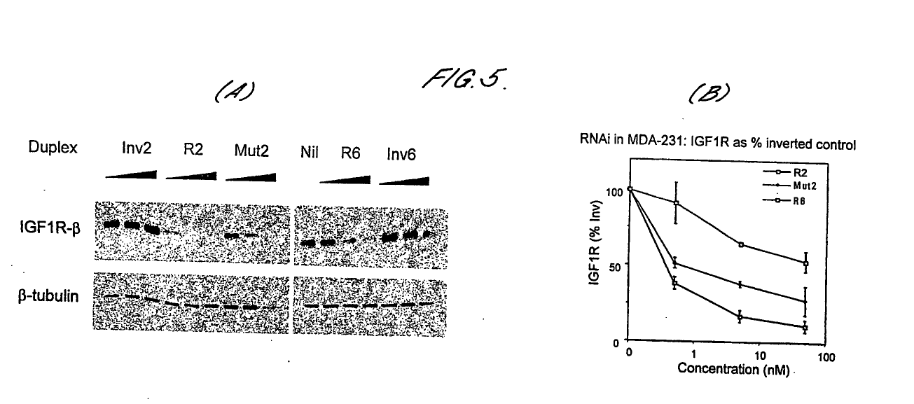

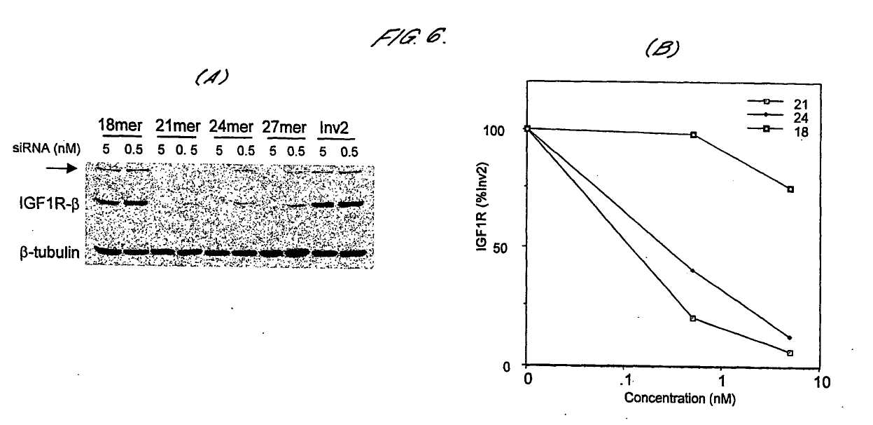

FIG. 7 A) MDA-MB-231 cells transfected with 100 nM RNA duplexes were serum-starved overnight and treated with 50 nM IGF-I for 30 min. Comparable results were seen in two sets of independently-prepared MDA-MB-231 cell lysates.
B) Clonogenic survival in MDA-MB-231 cells following transfection with 100 nM siRNA, inverted control duplexes (Inv), ASO4 or scrambled control (Scr4). Results represent mean±sem of colony counts in triplicate dishes. Similar results were obtained in two further clonogenic assays.
C) Clonogenic survival of transfected MDA-MB-231 cells that were unirradiated or irradiated at 2Gy. The fraction surviving 2Gy irradiation (SF2) is shown as mean±sem of three independent assays.

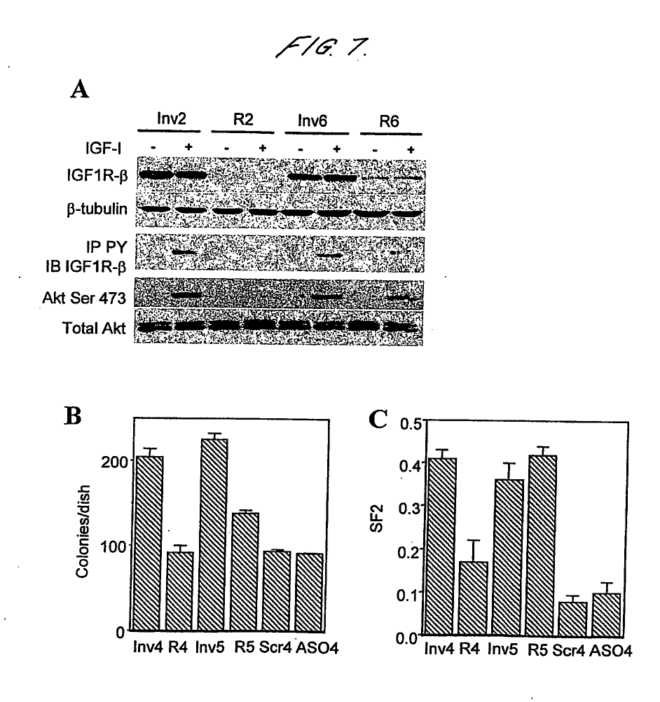

FIG. 8 illustrates growth in vivo in C57BL mice of B16 melanoma cells transfected with RNA duplexes. B16 melanoma cells were transfected with 200 nM duplexes (RNA22 is R2, Inv22 is Inv2) or oligofectamine alone (OF) for three consecutive days. On the 4th day cells were injected into the flanks of C57BL mice, using 2.5×10⁵ cells/mouse, using groups of 5 mice. Tumours were measured in 2 dimensions every 2-3 days. Tumour volumes were calculated as n(a×b²)/6 where a is the larger and b the smaller dimension.

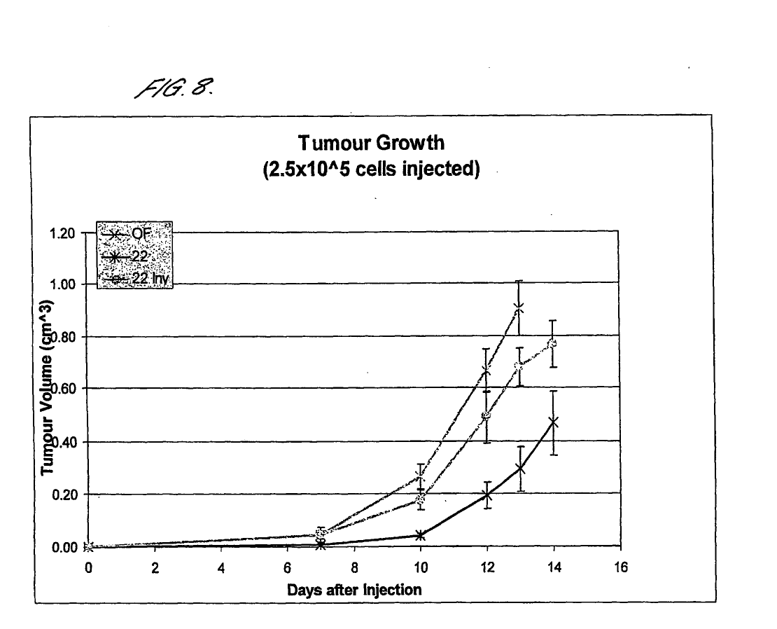

FIG. 9 shows the complete cDNA sequence for human IGF1R. The region evaluated using the scanning array is underlined.

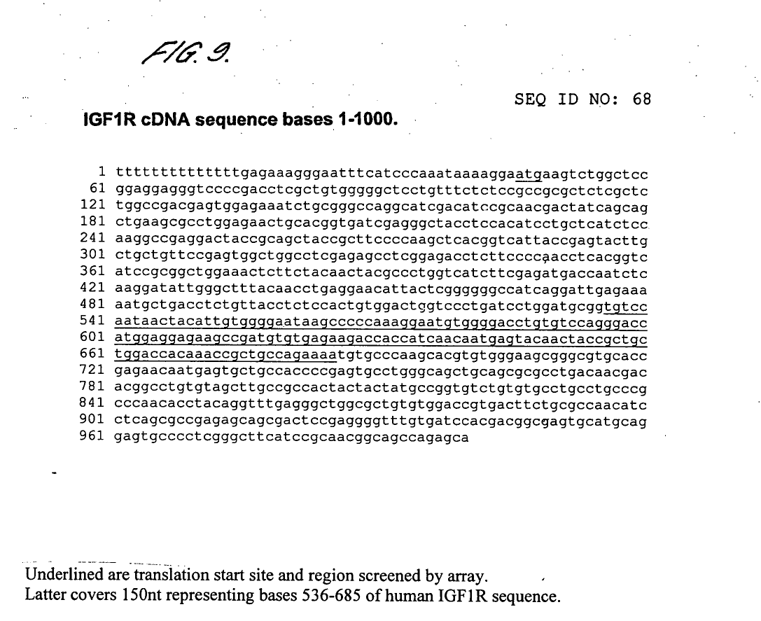

FIG. 10 illustrates the efficacy of 18mer antisense oligonucleotides in causing IGF1R downregulation in UC101 ovarian cancer cells. Results are expressed as % IGF1R expression in cells transfected with a scrambled control sequence.

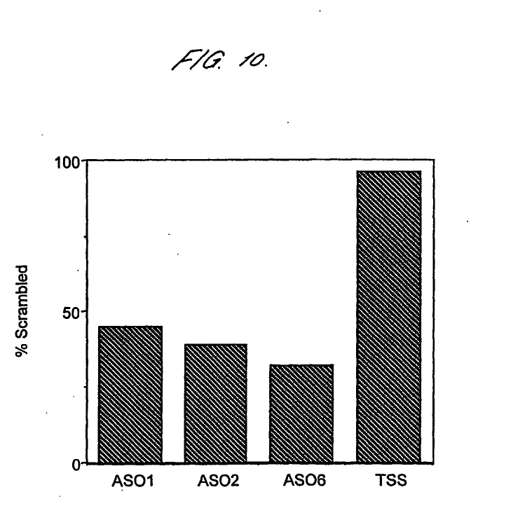

FIG. 11 illustrates how a small sequence shift may have a major effect on hybridization intensity and siRNA efficacy.
A) Phosphorimager analysis of peak 4 region of hybridization of IGF1R mRNA to 19mer IGF1R ASOs on the scanning array. ASOs equivalent to duplexes R4 and R5 are marked by arrowheads, and by black bars in the histogram.
B) Table shows hybridization intensity (arbitrary units, from scanning array) of 19mer ASOs equivalent to duplexes R2, R4, R5 and R6.
C) Representative immunoblot showing effects of 10 nM siRNAs or inverted controls on IGF1R levels in human DU145 prostate cancer cells.
D) Results (mean±sem) of triplicate analyses of IGF1R protein levels in DU145 prostate cancer and UC101 ovarian cancer cells transfected with 10 nM R4 or R5.

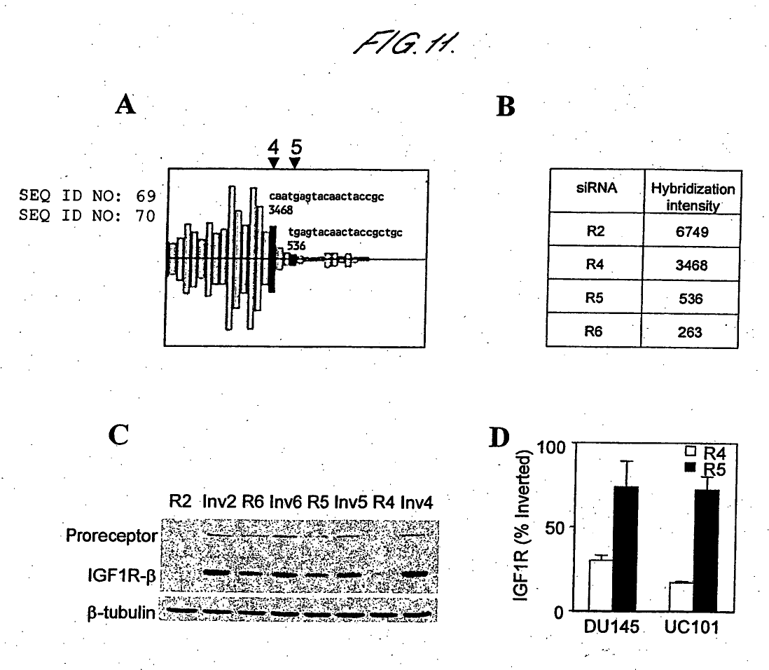

FIG. 12 illustrates growth of A549 cells under anchorage-independent conditions, in 24-well plates coated with polyHema to prevent adherence. Cells were transfected with 200 nM duplex, re-seeded the next day into polyHema-coated plates at 500 cells/well. MTS assay performed 10 days later. UT=untransfected. **p<0.01 for comparison of R4 with Scr.

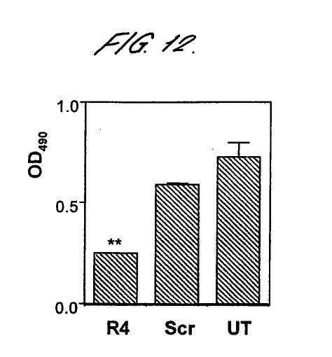

FIG. 13 illustrates the effect of siRNA R4 on apoptosis in prostate cancer cells. DU145 cells were transfected with 200 nM R4 or scrambled control, and after 48 hr were treated with etoposide or vehicle without drug. After overnight incubation at 37° C. the incidence of apoptosis was measured by caspase 3/7 activation assay (Promega; left panel), or by flow cytometry after staining with annexin V and propodium iodide (right panel). Table shows % of cells in early apoptosis (annexin positive, propidium negative).

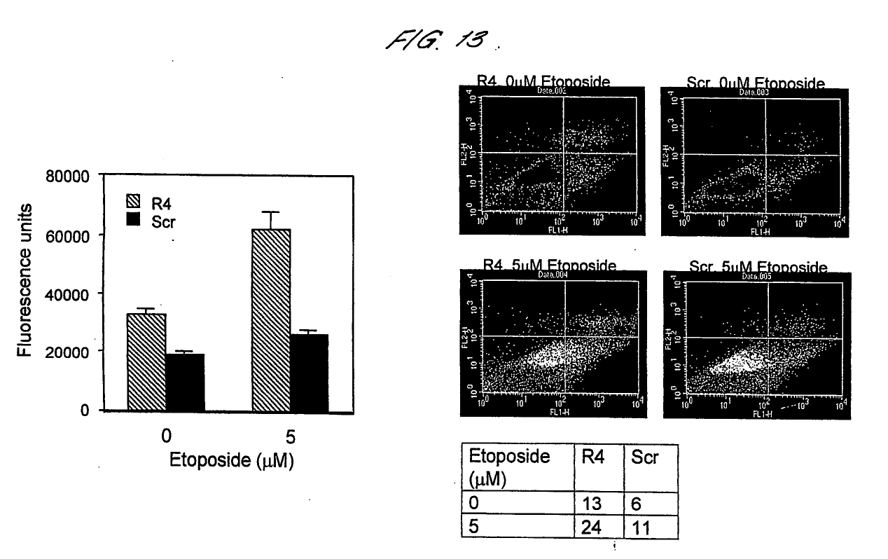

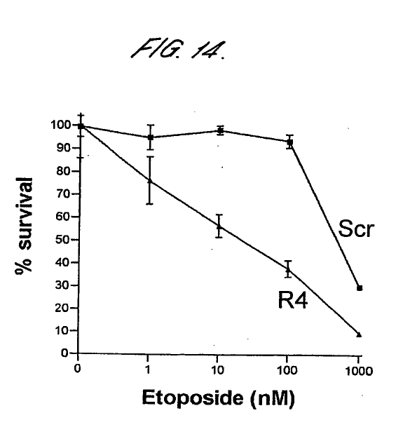

FIG. 14 illustrates chemosensitisation of MDA-MB-231 cells to etoposide. MDA-MB-231 cells were transfected with 200 nM siRNA R4 or scrambled control duplex, re-seeded the following day at 2000 cells/6 cm dish, and the following day treated overnight with etoposide at the indicated concentrations. Thereafter the drug was washed off, dishes were incubated for 1 week at 37° C., and visible colonies were stained and counted. Graph shows survival as % colony count in untreated dishes.

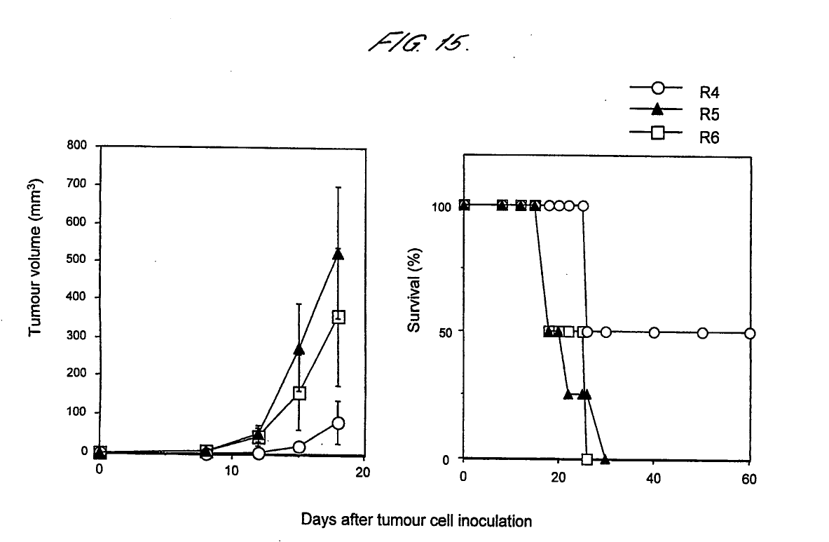

FIG. 15 illustrates growth of ovarian cancer xenografts following transfection in vitro with IGF1R siRNAs 4, 5 or 6.

UC101 ovarian cancer cells were transfected with 200 nM siRNA, mixed with irradiated NIH-3T3 cells and inoculated into the flanks of female athymic (nu/nu mice).

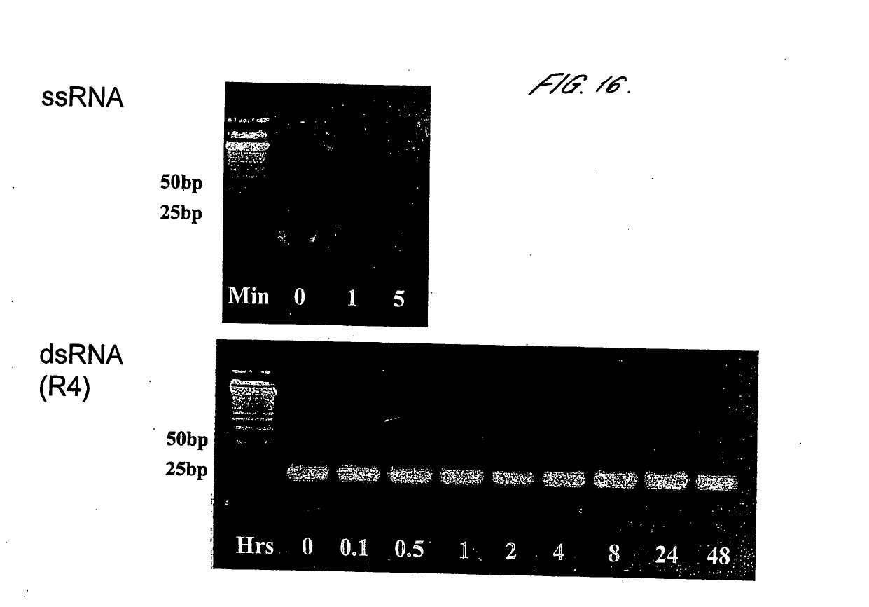

FIG. 16 illustrates the stability of siRNA R4 in foetal calf serum at 37° C., in comparison to a single-stranded RNA.

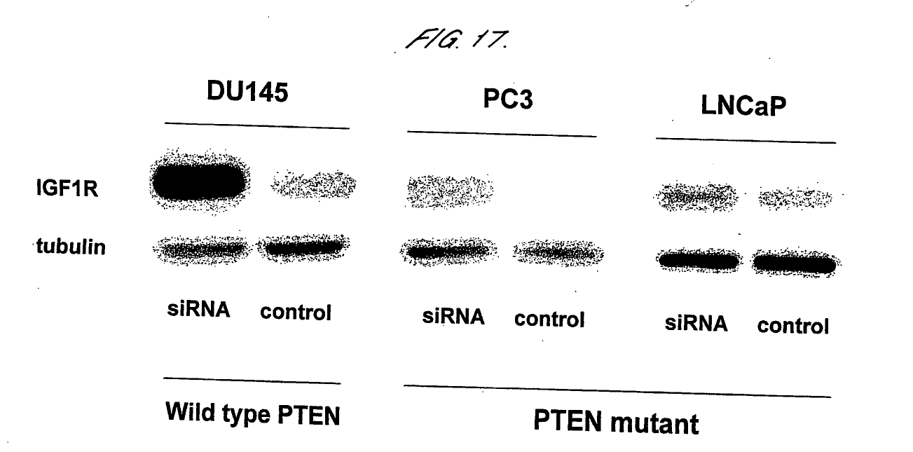

FIG. 17 shows how siRNA TSS downregulates the IGF1R in prostate cancer cells. Prostate cancer cells were transfected with 200 nM siRNA TSS homologous to nt 166-186 of the IGF1R gene or inverted sequence control. Western blot shows IGF1R levels 48 hours after transfection, indicating downregulation of IGF1R levels in cells treated with siRNA TSS in DU145 to 18%, in PC3 to 22%, in LNCaP to 17% of levels in cells treated with control duplex.

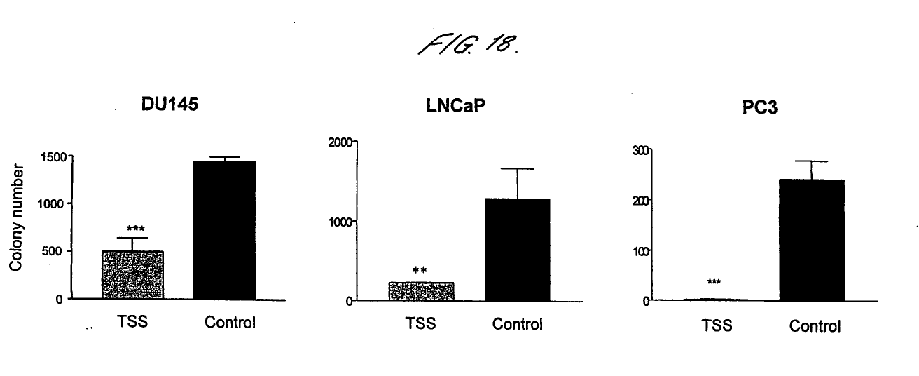

FIG. 18 illustrates how IGF1R gene silencing inhibits survival of prostate cancer cells. Human prostate cancer cells were transfected with 200 nM TSS or control duplex. After 48 hr the cells were disaggregated and re-seeded in 10 cm dishes (2000 cells/dish). After 1-3 weeks incubation (depending on the cell line), visible colonies were stained and counted. Bars show mean±sem of triplicate dishes. **p<0.01, ***p<0.001 for comparison of survival in cultures treated with TSS versus control duplex.

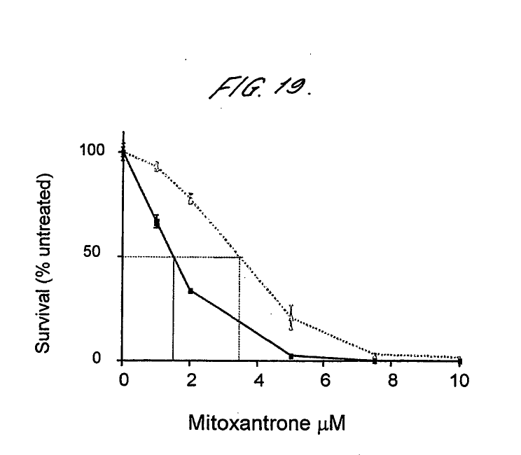

FIG. 19 illustrates that IGF1R gene silencing enhances chemosensitivity of androgen-resistant prostate cancer. PC3 cells were transfected with 200 nM TSS (red line) or scrambled sequence control duplex (green). After 48 hr the cells were disaggregated and re-seeded in 10 cm dishes (4000 cells/dish). The following day the cultures were treated with mitoxantrone at the indicated concentrations for 24 hours, after which the medium was changed and cells were allowed to survive to form colonies (>50 cells). Dotted lines mark IC₅₀₁ dose required to inhibit survival to 50% untreated level.

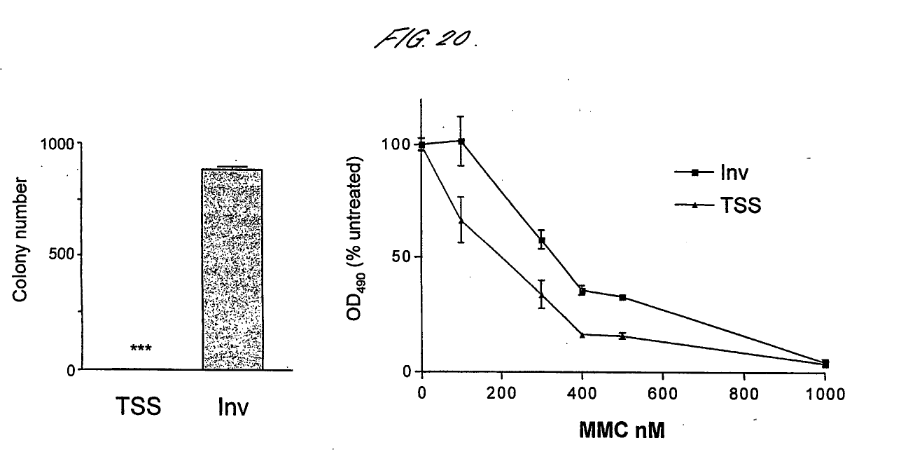

FIG. 20 illustrates that IGF1R gene silencing inhibits survival and enhances chemosensitivity of invasive bladder cancer. Human EJ28 invasive bladder carcinoma cells were transfected with 200 nM TSS or control duplex. After 48 hr the cells were disaggregated and re-seeded. To measure survival, 2000 cells were seeded in 10 cm dishes. Survival in TSS-transfected cells was only 0.2% of that in control-treated cultures, p<0.001). For chemosensitivity testing, cells were re-seeded into 96-well plates. The following day cultures were treated with mitomycin-C at the indicated concentrations for 24 hours, after which the medium was changed. After a further 4 days, growth was measured by calorimetric assay (MTS assay).

### EXAMPLE 1

### RNase H Mapping of IGF1R mRNA

RNase mapping was carried out in order to identify regions in the IGF1R mRNA which are accessible to RNaseH-mediated cleavage.

End labelled IGF1R mRNA was incubated with RNaseH and a chemically-synthesised library of 12mer oligonucleotides, according to the standard method described in Sohail, M. et al., Nucleic Acids Research, 2001, Vol. 29, pp 2041-2051.

Template Preparation:

Human IGF1R cDNA in plasmid pCVN was a generous gift from Renato Baserga. The 5′ region of IGF1R cDNA was cloned into vector pBluescript KS-(Stratagene) using restriction sites HindIII (pCVN-derived site at 5′ end of IGF1R cDNA) and Asp718 (cuts IGF1R cDNA at position 1581). This construct (template 1, 1.6 kb) included approximately 100 bp of polylinker sequence between the T7 promoter and the start of the IGF1R sequence. It was thought that this extraneous vector-derived sequence might influence folding of the transcript. Therefore this region was shortened by restriction digest using NotI, filling in with Klenow enzyme, digestion with EcoRV and re-ligating to make construct 2 (1.6 kbA55). Both constructs were linearised at the 3′ end of the IGF1R insert using Asp718, to make templates for in vitro transcription. End-labelled transcripts 1 (1.6 kb) and 2 (1.6 kbΔ55) (see FIG. 1) were generated using T7 RNA polymerase and γ-³²P-GTP (Amersham Pharmacia).

FIG. 1 is an autoradiograph showing gel analysis of the cleaved products of 5′-end labelled IGF1R mRNA with RNase H in the presence of a library of random 12mer oligonucleotides. The region from 500-700 bp was identified as being particularly accessible to RNase-mediated cleavage.

### EXAMPLE 2

### Oligonucleotide Scanning Array

Scanning arrays complementary to the region of the IGF1R mRNA from position 537-685 were prepared using the standard techniques described in Southern E. M. et al., Nucleic Acids Res., 1994, 22(8): 1368-1373; and Sohail, M. and Southern, E. M. “Using oligonucleotide scanning arrays to find effective antisense reagents”, Methods in Molecular Biology, vol. 170: DNA Arrays: Methods and Protocols, Ed J. B. Rampal, Humana Press Inc., Totowa, N.J.

Scanning arrays are a simple tool that allow combinatorial synthesis of a large number of oligonucleotides on a solid platform (typically glass or polypropylene, see note 1) in a spatially addressable fashion, and parallel measurement of the binding of all oligonucleotides complementary to the target mRNA.

The scanning arrays comprise sets of oligonucleotides of various lengths. A series of oligonucleotides, complementary to the target mRNA, is made by sequential coupling of nucleotides to a solid surface. The DNA synthesis reagents are applied to a confined area on the surface of the solid support using a mask. The mask is shifted along the surface after each round of coupling, resulting in a series of oligonucleotides each complementary to a region of the target sequence.

The scanning arrays will generally containing all complements of the selected target sequence up to a maximum length determined by the size of the template and template displacement used in the synthesis of the scanning array (Southern et al. 1994, ibid). In this instance the maximum length of the oligonucleotides in the array was either 18 or 20 nt, but this may be varied if required.

Detailed protocols for synthesis and probing of arrays are given below, in these protocols references to Figures denoted “Sohail and Southern” refer to the Figures of Sohail, M. and Southern, E. M. “Using oligonucleotide scanning arrays to find effective antisense reagents”, Methods in Molecular Biology, vol. 170: DNA Arrays: Methods and Protocols, Ed J. B. Rampal, Humana Press Inc., Totowa, N.J.:

Synthesis of a Scanning Array

Using a diamond-shaped or a circular reaction mask (Sohail and Southern, FIG. 1), it is possible to create arrays comprising sets of oligonucleotides of all lengths from monomers up to a maximum in a single series of couplings. The maximum length of oligonucleotides synthesised depends upon the ratio of the diagonal (for a diamond-shaped mask) or diameter (for a circular mask) of the mask to the displacement at each coupling step. For example, a diamond-shaped mask of 40 mm diagonal will produce 10-mers, 16-mers, or 20-mers using step sizes of 4 mm, 2.5 mm, or 2 mm, respectively. A diamond-shaped template creates a series of small diamond-shaped cells. The longest oligonucleotides are found along the centre line and the monomers are located at the edge (Sohail and Southern, FIG. 1). A circular template creates cells that differ in shape: along the centre line, they are lenticular, but off this line, they form a four-cornered ‘spearhead’ that diminishes in size towards the edge. The arrays as synthesised are symmetrical above and below the centre line of the template and each oligonucleotide is represented twice allowing for duplicate hybridisation measurements.

For each length of oligonucleotides s, there are N-s+1 s-mers covering a total length of N bases. For example, if a 150 nt long sequence is covered in a 150 step synthesis, there will be 150 monomers and 131 20-mers. The last 20 positions in the sequence will be represented by shorter oligonucleotides only; in this case, from 19-mer to monomer. Therefore, for making 200 20-mers, an additional 19 nt synthesis steps need to be added at the end, i.e., total coupling steps=N+s−1.

Materials

Derivatisation of Glass

1. Glass cylinder and apparatus shown in Sohail and Southern FIG. 3.
2. Glass sheets of required dimension (3 mm thick: Pilkington, UK).
3. 3-Glycidoxypropyl trimethyoxysilane (98% v/v: Aldrich).
4. Di-isopropylethylamine (99.5% v/v: Aldrich).
5. Xylene (AnalaR: Merck).
6. Hexaethylene glycol (97% v/v: Aldrich).
7. Sulfuric acid (AnalaR: Merck).
8. Ethanol (AnalaR: Merck).
9. Ether (AnalaR: Merck).
10. Water bath at 80° C.
   Making Reaction Masks
1. Stainless steel or aluminium square metal piece or PTFE (Teflon). Dimensions of the workpiece may vary according to the size of the mask.
2. A centre lathe or a horizontal milling machine.
3. A drilling machine.
4. Abrasive paper from ˜P600 to P1200 (3M Inc., USA) and polishing grade crocus paper (J. G. Naylor & Co. Ltd., Woodley, Stckport, Manchester, England).
   Making Scanning Arrays
1. Solid support (derivatised glass or aminated polypropylene (Beckman Coulter, Inc., USA)).
2. DNA synthesiser (ABI).
3. A reaction mask of desired shape and size and assembly frame (see Sohail and Southern FIG. 4 for the assembly).
4. DNA synthesis reagents: standard dA, dG, dC and T phosphoramidites, oxidizing agent, acetonitrile, activator solution, deblock solution (all from Cruachem).
5. Reverse phosphoramidites bought from Glen Research
   Deprotection of Arrays
1. Assembly for constructing deprotection bomb as shown in Sohail and Southern FIG. 5. The assembly consists of a high density polyethylene (HDPE) chamber, 4 mm thick silicon rubber gasket and a stainless steel plate of the dimensions of the HDPE chamber, and stainless steel M8 nuts and bolts.
2. 30% ammonia solution (AnalaR: Merck).
3. Water bath at 55° C.
   In Vitro Transcription
1. Template DNA (at ˜1 mg/mL).
2. T7 or SP6 RNA polymerase, transcription buffer (5× transcription buffer is, 200 mM Tris-HCl pH 7.9, 30 mM MgCl₂, 10 mM spermidine, 50 mM NaCl), recombinant RNAsin®, 100 mM DTT and nuclease-free distilled water (Promega).
3. [α-³²P] UTP (3000 Ci/mmol) or [α-³³P] UTP (2500 Ci/mmol) (Amersham).
4. rNTPs (Pharmacia): ATP, GTP, CTP stored as 10 mM solution, and UTP as 250 mM solution in nuclease-free distilled water. Store all reagents at −20° C.
   Quantitation of Transcripts
1. Scintillation counter (e.g., Beckman LS 1710).
2. Scintillation vials and scintillation fluid (Amersham).
   Hybridisation, Imaging and Analysis
1. Hybridisation buffer (1M NaCl, 10 mM Tris-HCl pH 7.4, 1 mM EDTA, 0.01% SDS (w/v)) (see Note. 2).
2. 50-100 fmol radiolabelled transcript.
3. A glass plate of the size of the array when using an array made on glass, a moist chamber (a large plastic or glass lidded box containing wetted paper towels) and an incubator set to the desired temperature.
4. A hybridisation tube and oven used in standard Southern hybridisation (e.g., Techne) when using an array made on polypropylene.
5. Esco rubber tubing of OD 1 mm (Sterlin) for use in Section 3.8.
6. Storage phosphor screen (Fuji or Kodak).
7. PhosphorImager or STORM (Molecular Dynamics).
8. A SUN Solaris work station for image analysis and the computer software xvseq (L. Wand and J. K. Elder, unpublished) (available by anonymous ftp at ftp://bioch.ox.ac.uk/pub/xvseq.tar.gz).
   Stripping of Arrays
1. Stripping solution (100 mM sodium carbonate/bicarbonate buffer pH 10, 0.01% SDS (w/v)) (see Note 3).
2. Geiger-Müller counter (Mini-Instruments Ltd.).
   Methods

Derivatisation of Glass
1. Prepare a mixture of di-isopropylethylamine, glycidoxypropyl trimethoxysilane and xylene (1: 17.8: 69, v/v/v) in a glass cylinder and completely immerse the glass plates in the mixture. Incubate as shown in Sohail and Southern FIG. 3 at 80° C. for 9 h.
2. Remove the plates, allow them to cool to room temperature, and wash with ethanol and then with ether by squirting the liquid from a wash bottle.
3. Incubate the plates in hexaethylene glycol containing a catalytic amount of sulfuric acid (˜25 mL/L) at 80° C. for 10 h, with stirring.
4. Remove the plates, allow them to cool to room temperature, and wash with ethanol and ether. Air dry and store at −20° C.
   Machining of Masks
1. Both stainless steel or aluminium can be used to make diamond-shaped and circular reaction masks. Circular masks are made using a centre lathe and diamond-shaped masks using a horizontal milling machine (see Note 4).
2. To make a diamond shaped mask from metal, hold the work piece at an angle of 45° to the axis of the bed of the milling machine (the diagonal of the diamond running parallel to the axis of the bed.
3. Machine the cavity to the required depth (generally between 0.5-0.75 mm) to create a reaction chamber. Machine the outer lands to a depth of approximately 0.5 mm to form the sealing edge (0.3-0.5 mm wide) (Sohail and Southern FIG. 4).
4. Using the smallest possible diameter cutter (˜1.5 mm), radius the internal corners of the reaction chamber.
5. Finish the sealing edge by polishing flat with successively finer grades of wetted abrasive paper (from ˜P600 to P1200) and finally with a polishing grade crocus paper.
6. Drill holes of 1.08 mm diameter for reagent inlet and outlet, respectively, at the bottom and the top of the reaction chamber (in the corners of the diamonds). Inlet and outlet connections to the DNA synthesiser are made using standard 19SWG syringe needles (1.1 mm diameter) with chamfered ends ground off and de-burred (see Note 5).
   Fabrication of Arrays
1. Cut glass or polypropylene to the correct size. The process of making an array is the same when using either glass or polypropylene. Polypropylene has to be mounted on a glass plate, e.g., 3 mm thick soda glass (see Note 6). The total area covered by an array for N bases using a mask of diagonal or diameter D mm and step size 1 mm is N×1+D mm. 2-3 mm are added to margins to allow easy manipulations.
2. Fix the assembly (Sohail and Southern FIG. 4) to the front of a DNA synthesiser and connect its inlet and outlet to the synthesiser's reagent supply.
3. Program the DNA synthesiser with an appropriate synthesis cycle. A slightly modified cycle is used, for example, the one given in Table 1 (Sohail and Southern). Also check all the reagent bottles.
4. Enter the sequence (antisense strand) in 5′ to 3′ direction.
5. Mark the first footprint of the reaction mask on the support by placing it against the mask on the assembly in the desired starting position (see Sohail and Southern FIG. 6). A knife is used to make notches in polypropylene. A diamond scriber can be used to mark glass.
6. Tighten the plate against the mask with the pressure clamp to produce a seal (Sohail and Southern FIG. 4b). Sufficient pressure is applied to stop leakage (˜500-800 Newton force) but not enough to create indentations in the polypropylene surface which can lead to leakage of reagent from the mask during subsequent synthesis steps.
7. Start the DNA synthesiser to go through the pre-programmed cycle to couple the appropriate nucleotide. The first condensation on the substrate is of base at the 3′ end of the sequence.
8. After completion of the step during the interrupt (see Sohail and Southern Table 1 and Note 7), slacken the pressure clamp and move the plate one increment (Sohail and Southern FIG. 4b).
9. Tighten the pressure clamp and start the synthesiser for the next nucleotide in the sequence. Continue the process until the full sequence length is synthesised (see Note 8).
   Deprotection of Arrays
1. Place the glass or polypropylene array(s) into the HDPE chamber (see Note 9) and add 30% ammonia into the chamber to cover the array(s) completely.
2. Place the silicon rubber gasket around the rim of the chamber and the stainless steel plate on top of the gasket.
3. Place bolts through the metal plate, the gasket and the HDPE chamber, and tighten.
4. Incubate in a water bath at 55° C. for 12-18 h in a fume hood.
5. Cool the assembly to 4° C. before opening. The arrays are ready to be used in hybridisation at this stage.
   Preparing and Quantifying Radiolabelled Transcripts

1. Set an in vitro transcription reaction (20 μL) by adding the following components to a microfuge tube at room temperature.

| | | |
|---|---|---|
| 5 × transcription buffer | 4 | μL |
| 100 mM DTT | 2 | μL |
| RNAsin ™ | 20 | U |
| 10 mM ATP, GTP, and CTP | 1 | μL each |
| 250 mM UTP | 1 | μL |
| Template DNA | 2-3 | μL (see Note 10) |
| [α-32P]UTP or [α-33P]UTP | 2 | μL |
| T7 or SP6 RNA polymerase | 20 | U |
| Total Volume | 20 | μL |

Mix and incubate at 37° C. for 1 h.

2. Remove 1 μL for quantitation (see below).
3. Remove unincorporated label by Sephadex® G25 column chromatography (see Note 11).
4. Save 1 μL of the purified transcript for quantitation (see below).
5. Check the integrity of the transcript by denaturing polyacrylamide gel electrophoresis (10).
6. Add 10 μL of the scintillation fluid to the samples saved in Steps 2 and 4.
7. Mix by vortexing and count the samples in a scintillation counter for 1 min.
8. Calculate the percent incorporation:

$$\% incorporation = \frac{incorporated cpm \times 100}{total cpm}$$
9. Calculate the amount of RNA made:

$$\begin{matrix}Amount of [ α {-}^{32} P ] UTP = \frac{20 µCi}{3000 µCi \text{/} n mol} \\ = 6.6 \times {10}^{- 3} mol \\ Amount of cold UTP = 1 µL \times 250 µM \\ = 0.250 n mol \\ Total UTP = 6.6 \times {10}^{- 3} + 0.25 \\ = 0.256 n mol \\ \begin{matrix}For a reaction with 50 \% \\ incorporation , the amount \\ of UTP incorporated\end{matrix} = 0.256 n mol \div 2 \\ = 0.128 n mol \\ \begin{matrix}Supposing equal incorporation \\ of all four nucleotides , \\ total nucleotides incorporated\end{matrix} = 0.128 n mol \times 4 \\ = 0.512 n mol \\ Amount of full \text{-} length transcript = 0.512 \div total length of transcript\end{matrix}$$

Hybridisation to Arrays Made on Polypropylene
1. Place the array in the hybridisation tube, coiling it in a spiral.
2. Dilute the radiolabelled transcript in an appropriate volume (10-20 mL depending upon the size of the array and the hybridisation tube) of hybridisation buffer. The mix should cover the array along the length of the tube.
3. Place items 1 and 2 in the oven at desired temperature for 30 min. Also put approximately 100 mL of the hybridisation buffer in the oven: this is to be used to wash the array at the end of hybridisation.
4. Pour the hybridisation mix into the tube containing the array and hybridise for 3-4 h.
5. Remove the hybridisation mix. Briefly wash the array with the hybridisation buffer from Step 3, air dry, cover with cling film and expose to a storage phosphor screen for 16-20 h (see Note 12).
6. Scan the screen on PhosphorImager or STORM and analyse the image using xvseq (see below).
   Hybridisation to Arrays Made on Glass
1. Clean the non-array glass plate with acetone and ethanol to ensure it is grease-free and siliconise it by treatment with dimethyl dichlorosilane solution and place it in lidded box. Also place moist paper towel in the box.
2. Dilute the radiolabelled transcript in an appropriate volume of the hybridisation buffer (for example, for an array of 250 mm×50 mm use 500-750 μL).
3. Place items 1 and 2 in an incubator at desired temprature for 30 min. Also put approximately 100 mL of the hybridisation buffer in the oven: this is to be used to wash the array at the end of hybridisation.
4. Using a micropipette, pipette the hybridisation mix in a line evenly along the length of the non-array glass plate, avoiding formation of air bubbles.
5. Starting at one end, carefully place the scanning array (face down) on top of the hybridisation mix. The mix will spread out and form a thin film between the two plates. Incubate for 3-4 h.
6. Separate the plates from each other and wash the array plate with hybridisation buffer to remove unbound mix. Drain the plate, air dry, cover with cling film and expose to a storage phosphor screen for 16-20 h.
7. Scan the screen on PhosphorImager or STORM and analyse the image using xvseq (see below).
   Alternative Hybridisation Protocol for Glass or Polypropylene
1. Assemble with clips the array plate (or polypropylene array pasted with PhotoMount™ on a glass plate) and the non-array plate, using rubber tubing as spacers on two sides.
2. Dilute the radiolabelled transcript in approximately 5-10 mL of hybridisation buffer.
3. Follow Step 3, Protocol 3.7.
4. Inject the hybridisation mix into the space between the two plates with a needle and syringe.
5. Incubate the assembly in horizontal position at desired temperature.
6. Follow Steps 6 and 7, Protocol 3.7.
   Image Analysis

The hybridisation images are analysed using xvseq (see Sohail and Southern FIG. 7). This program reads and displays images generated by a PhosphorImager or STORM and can also perform standard image manipulation such as scaling, clipping and rotation. Although visual inspection of an image reveals the results generally, computer-aided analysis is needed to obtain quantitative information about hybridisation intensities and the oligonucleotide sequences that generated them. xvseq calculates and displays integrated intensities of the array oligonucleotides, each of which corresponds to an image cell formed by intersection of overlapping array templates.

The user can specify the template size, shape and location, step size between successive templates, as well as the sequence that was used to make the array. The template grid is superimposed on the image and the template parameters are adjusted interactively to achieve correct and accurate registration of the grid with the hybridisation pattern. It can be difficult to achieve precise registration by reference to the hybridisation pattern alone, especially, if the signals at either edge of the array are weak or undetectable. Avoid placing the template grid so that it appears to be registered but is in fact misaligned by one or more template steps. Registration can be aided by the use of fixed reference points on an array such as those shown in Sohail and Southern FIG. 6.

Stripping of Arrays

1. The arrays can be used several times. To strip, heat an appropriate volume of the stripping solution to 90° C. in a glass beaker.
2. Immerse the array in the hot stripping solution and stir for 1-2 min.
3. Remove the array and monitor with a Geiger counter to confirm that most of the radiolabel has been removed. Repeat steps 1-2 if radioactivity on the surface of the array is still detectable.
4. Allow the array to cool down to room temperature and wash it thoroughly with nuclease-free distilled water, 70% (v/v) ethanol and finally with absolute ethanol.
5. Air dry and store the array at −20° C. until future use.
   Notes
1. The choice of array substrate material and attachment chemistry is important for making high quality arrays. A flat, impermeable surface is required for in situ synthesis of arrays. Glass has a number of favourable qualities, including its wide availability, smooth surface, transparency, chemical stability and compatibility with the use of both radiolabelled or fluorescence labelled nucleic acids targets. Glass is chemically derivatised as described in the methods section to produce a hexaethylene glycol linker which has a terminal —OH group that allows condensation of nucleotide phosphoramidites (Maskos, U., and Southern, E. M. (1992) Nucleic Acids Res. 20, 1679-1684.). Similarly, polypropylene also has favourable physical and chemical properties. Polypropylene is aminated to produce amine groups (Matson, R. S., Rampal, J. B., and Coassin, P. J. (1994) Anal. Biochem. 217, 306-310.) that also allow synthesis to oligonucleotides using standard CE nucleotide phosphoramidites.

For the array fabrication method described here, it is important that a tight seal is formed between the substrate material and the reaction mask. Metals form tight seal with polypropylene but not with glass. PTFE seals well against both glass and polypropylene.
2. 1M NaCl is used routinely. Alternative buffers are: (i) 1 M NaCl, 5-10 mM MgCl₂, 10 mM Tris-HCl pH 7.4, 1 mM EDTA, 0.01% SDS (w/v), and (ii) 150 mM NaCl, 10 mM MgCl₂, 10 mM Tris-HCl pH 7.4, 1 mM EDTA, 0.01% SDS (w/v).
3. Addition of more than 0.01% SDS can damage arrays.
4. Circular masks can also be made from PTFE (Teflon). Diamond-shaped masks are more difficult to make with PTFE by the machining process but can be made by pressure molding in a hydraulic press (−150 ton force) using a pre-machined die.
5. Holes should be made as close as reasonably possible to the sealing edge without damaging it. Care must be taken to de-burr fully the holes at the point of entry into the reaction chamber. For PTFE masks the holes should be 1.0 mm diameter which make virtually 100% leak-tight seal. In the case of metal masks, the 0.02 mm interface indicated above also provides a leak-tight seal without the use of any additional sealer. Care must be taken not to insert the end of the syringe needle into the reaction chamber void.
6. Unlike glass, polypropylene is not rigid and thus needs to be mounted on a solid, flat surface for its precise movement against the reaction mask during synthesis. Even mounting of polypropylene on glass is important to produce a good seal between the sealing edge of the reaction mask and the polypropylene surface. Glass used must be clean and free from dust particles because they can cause bulging of the polypropylene which can hinder the formation of a proper seal. A very thin layer of PhotoMount™ (3M Inc, USA) which can be used to paste polypropylene to glass, should be sprayed on glass and not polypropylene.
7. At the start of each synthesis cycle, an interrupt step can be introduced to halt the process at the first step of the next nucleotide condensation cycle to allow the operator to move the plate and restart the program. Alternatively, a long wait step at the beginning of the program can be introduced (see Sohail and Southern Table 1) if the operator does not wish to use the interrupt step. The operator is also advised to consult the user's manual for the DNA synthesiser.
8. With the use of standard phosphoramidites in the synthesis, the oligonucleotides are attached to the solid support at their 3′ ends. Reverse phosphoramidites can be used to make oligonucleotides that are attached at their 5′ ends.

Iodine is used as an oxidising agent. At lower temperatures it will take longer to reach the top of the reaction cell. Iodine can also be replaced with sulfurising agent (Cruachem) to make arrays of phosphorothioate oligonucleotides.
9. When using the standard phosphoramidites, the exocyclic amines of the bases are protected chemically to prevent side reactions during synthesis. These protecting groups need to be removed from the coupled bases before hybridisation.

Before deprotection, detach the polypropylene arrays from glass by peeling from one end. PhotoMount can be removed with ethanol, acetone or dichloromethane.
10. An internally radiolabelled RNA is used as target to hybridise to a scanning array which is generated by in vitro transcription, carried out in the presence of [α-³²P]UTP or [α-³³P]UTP (or [α-³²P]CTP) using an appropriate DNA template. A plasmid containing the desired DNA fragment under the transcriptional control of a T7 or SP6 promoter (such as pGEM: Promega) can be used as template. The plasmid is linearised with an appropriate restriction endonuclease to produce transcripts of defined length without contaminating vector sequence. Alternatively, a template with T7 or SP6 RNA promoter can also be generated using the polymerase chain reaction: primers are used to amplify the required fragment from a plasmid, genomic DNA or cDNA, such that the sense primer has a T7 or SP6 promoter leader sequence. (Sohail and Southern Table 2) added at the 5′ end.
11. Sephadex® G25 columns are available from several commercial suppliers including Promega and Pharmacia. Spin columns made in-house, as described in (Sambrook, J., Fritsch, E. F., and Maniatis, T. (1989) Molecular Cloning: A Laboratory Manual. Cold Spring Harbor, N.Y.), can also be used.
12. For hybridisation below 37° C., care must be taken not to touch the plates because this can lead to melting of short duplexes. For hybridisation below room temperature, the cling film and the phosphor screen must be cooled to hybridisation temperature and exposed at the same temperature.

In this study hybridisation to the arrays was carried out at physiological temperature (37° C.) in addition to room temperature (23° C.), in order to select sequences which are more likely to have activity in intact cells. The arrays were probed with labelled IGF1R mRNA, and also with labelled insulin receptor mRNA in order to identify oligonucleotides which have high specificity for IGF1R mRNA.

Results

Representative plots of hybridisation intensity across the array for 15mers and 18mers probed with labelled IGF1R mRNA are shown in FIG. 2. The plots are annotated to show the position of selected individual oligonucleotide sequences. In each case the sequence given corresponds to the sense strand of the IGF1R mRNA.

Relative hybridisation intensities for selected oligonucleotides were calculated relative to the most strongly hybridising sequence. For comparison purposes the 15mer sequence described by Wraight et al. which falls within the region 537-685 is included:

| | | | |
|---|---|---|---|
| | | Hyb | Rel hyb |
| Oligo | Sequence | intensity | intensity |

| | | | |
|---|---|---|---|
| ASO2 | ggtcttctcacacatcgg | 56,026 | 1.00 |
| ASO4 | gcggtagttgtactcattgt | >33,162 | >0.59 |
| ASO1 | ggcttctcctccatggtc | 15583 | 0.28 |
| ASO3 | attgttgatggtggtcttct | >7048 | >0.13 |
| ASO5 | gtggtccagcagcggtagtt | >4150 | >0.07 |
| ASO6 | cttattccccacaatgta | 560 | 0.01 |
| 15mer | gtgcagcagcggtag | 1695 | 0.03 |

### EXAMPLE 4

### Synthesis of Antisense Oligonucleotides and RNAi Duplexes

Based on the results of array screening, 18-20mer IGF1R antisense oligonucleotides (ASOs) which hybridised strongly to IGF1R transcript were selected for further study. The ASOs were synthesised as phosphorothioates and HPLC purified by the Cancer Research UK oligonucleotide synthesis service, Clare Hall, Hertfordshire, UK. Selected ASO sequences are listed in table 1. ASOs 1, 2, 4, and 5 hybridised strongly to IGF1R mRNA at 37° C. on the basis of the scanning array results. ASO6 was included for comparison because it hybridises poorly to IGF1R mRNA on the basis of the array screening results, AS03 was also included for comparison purposes because it hybridises only at 23° C. TSS is complementary to the human IGF1R translation initiation site, and is identical to that described by Baserga et al., (U.S. Pat. Nos. 6,340,674 and 5,643,788) and Resnicoff et al., 1994. For each ASO a contol scrambled sequence oligonucleotide (Scr) containing the same bases in a random, non-homologous sequence was also synthesised. Lyophilised oligonucleotides were reconstituted in sterile TE (10 mM Tris-Cl, 1 mM EDTA).

TABLE 1
Phosphorothioate ASO sequences

| | | |
|---|---|---|
| ASO1 (18mer) | GGCTTCTCCTCCATGGTC | SEQ ID NO:1 |
| ASO1 (20mer) | CGGCTTCTCCTCCATGGTCC | SEQ ID NO:2 |
| Mouse AS01 | GGGCTTCTCCTCCAATGTCC | SEQ ID NO:3 |
| Scr1 | TCTTCCGCGACTTGCTCCGC | SEQ ID NO:4 |
| Mouse Scr1 | CTGTTCACCGTGCACCCTGT | SEQ ID NO:5 |
| ASO2 (18mer) | GGTCTTCTCACACATCGG | SEQ ID NO:6 |
| ASO2 (20mer) | TGGTCTTCTCACACATCGGC | SEQ ID NO:7 |
| Mouse ASO2 | TGGTCTTCTCACACATGGGC | SEQ ID NO:8 |
| Scr2 | CCTGTACGCGTTGATCTCCA | SEQ ID NO:9 |
| Mouse Scr2 | AGTCGCCTAGTCGAGTCCTT | SEQ ID NO:1O |
| ASO3 | ATTGTTGATGGTGGTCTTCT | SEQ ID NO:11 |
| Scr3 | TCATGCTTGTTGATGTGGTT | SEQ ID NO:12 |
| ASO4 | GCGGTAGTTGTACTCATTGT | SEQ ID NO:13 |
| Scr4 | TGTCGTTCGAGTTGATCGTA | SEQ ID NO:14 |
| ASO5 | GTGGTCCAGCAGCGGTAGTT | SEQ ID NO:15 |
| Scr5 | GACGTTAGCGTGCGATGTGC | SEQ ID NO:16 |
| ASO6 | CTTATTCCCCACAATGTA | SEQ ID NO:17 |
| Mouse ASO6 | CTTGTTCCCCACAATGTA | SEQ ID NO:18 |
| Scr6 | CATAGCACATTCTTCTCA | SEQ ID NO:19 |
| Mouse Scr6 | ACCGTTCATCAGTTCACT | SEQ ID NO:20 |
| TSS | TCCTCCGGAGCCAGACTT | SEQ ID NO:21 |
| ScrTSS | CAGCTACTCGCATGCTGC | SEQ ID NO:22 |

Double-stranded RNA duplexes for RNAi corresponding in sequence to certain of the ASOs were also synthesised. RNA oligonucleotides were synthesised and HPLC purified at Cruachem, Glasgow. Lyophilised oligoribonucleotides were reconstituted in nuclease-free water and diluted to 50 μM. Complementary strands were annealed in 100 mM potassium acetate, 30 mM Hepes-KOH pH 7.4, 2 mM magnesium acetate, as described (Elbashir et al., 2001, www.dharmacon.com) to give a final concentration of 20 μM duplex. Duplex formation was checked by electrophoresis through 5% low-melting point agarose (NuSieve GTG, BioWhittaker Molecular Applications) in 1×TBE.

TABLE 2
RNAi duplex sequences

| | | |
|---|---|---|
| R2 | GCCGAUGUGUGAGAAGACCTT | SEQ ID NO:23 |
| | TTCGGCUACACACUCUUCUGG | |
| R2 | GCCGAUGUGUGAGAAGTT | SEQ ID NO:24 |
| (18mer) | TTCGGCUACACACUCUUC | |
| R2 | GCCGAUGUGUGAGAAGACCACCTT | SEQ ID NO:25 |
| (24mer) | TTCGGCUACACACUCUUCUGGUGG | |
| R2 | GCCGAUGUGUGAGAAGACCACCAUCTT | SEQ ID NO:26 |
| (27mer) | TTCGGCUACACACUCUUCUGGUGGUAG | |
| Inv2 | CCAGAAGAGUGUGUAGCCGTT | SEQ ID NO:27 |
| | TTGGUCUUCUCACACAUCGGC | |
| Mut2 | GCCGAUGUGUGUGAAGACCTT | SEQ ID NO:28 |
| | TTCGGCUACACACACUUCUGG | |
| MouseR2 | GCCCAUGUGUGAGAAGACCTT | SEQ ID NO:29 |
| | TTCGGGUACACACUCUUCUGG | |
| R4 | CAAUGAGUACAACUACCGCTT | SEQ ID NO:30 |
| | TTGUUACUCAUGUUGAUGGCG | |
| Inv4 | CGCCAUCAACAUGAGUAACTT | SEQ ID NO:31 |
| | TTGCGGUAGUUGUACUCAUUG | |
| R5 | UGAGUACAACUACCGCUGCTT | SEQ ID NO:32 |
| | TTACUCAUGUUGAUGGCGACG | |
| Inv5 | CGUCGCCAUCAACAUGAGUTT | SEQ ID NO:33 |
| | TTGCAGCGGUAGUUGUACUCA | |
| R6 | CUACAUUGUGGGGAAUAAGTT | SEQ ID NO:34 |
| | TTGAUGUAACACCCCUUAUUC | |
| Inv6 | GAAUAAGGGGUGUUACAUCTT | SEQ ID NO:35 |
| | TTCUUAUUCCCCACAAUGUAG | |
| MouseR6 | CUACAUUGUGGGGAACAAGTT | SEQ ID NO:36 |
| | TTGAUGUAACACCCCUUGUUC | |
| siRNA | CGACUAUCAGCAGCUGAAGTT | SEQ ID NO:37 |
| TSS | TTGCUGAUAGUCGUCGACUUC | |

### EXAMPLE 5

### Activity of ASOs and RNAi Duplexes in Mammalian Cell Lines

Cell Culture and Transfection

The human and murine cell lines used in this study were cultured in RPMI 1640 plus 10% FCS at 37° C. in a humidified atmosphere of 5% CO₂. The cultures were negative when tested for Mycoplasma infection. Cultures were passaged the day before transfection to achieve 30-50% confluence the following day. Initial transfections of ASOs used 1-2.5 μg/ml Cytofectin (Glen Research, Sterling, Va.), according to the manufacturer's instructions. Latterly Oligofectamine (Life Sciences) has been used for all ASO and RNAi transfections. The method was as described (see manufacturer's instructions and www.dharmacon.com) with minor modifications. For transfection of monolayers seeded in 6 well plates, 5 μl phosphorothioate oligonucleotide or RNA duplex at 100× final concentration were mixed with 2501 serum-free Optimem (Gibco-BRL). In a separate tube 2 μl Oligofectamine was mixed with 68 μl Optimem. After 10 min incubation at room temperature the contents of the two tubes were mixed and incubated for a further 25 min at room temperature. Monolayers were washed with 1-2 ml Optimem. To the cells were added 175 μl Optimem followed by the 325 μl complexes. Volumes were scaled up by factors of 2.22 or 6.05 for transfection in 60 mm or 100 mm dishes respectively. Cultures were incubated at 37° C. for 4 hr, and then 50 μl FCS and 3.5 ml RPMI plus 5% FCS were added to each well. After 48 hr incubation effects on IGF1R and IR expression were determined by immunoblotting. Some transfected cultures were serum-starved overnight, divided into two aliquots and treated with 10 nM Long R3-IGF-I (GroPep, Adelaide) or diluent for 30 min at 37° C. prior to lysis.

Immunoblotting

IGF1R expression was assessed by immunoblotting as previously described (Macaulay et al 2001). After washing in ice-cold PBS, cells were lysed in 50 mM Hepes pH 7.5, 100 mM NaCl, 10 mM EDTA, 1% Triton-X-100, 4 mM sodium pyrophosphate, 2 mM sodium orthovanadate, 10 mM sodium fluoride, 1 mM PMSF, 2 μg/ml each leupeptin and aprotinin. Lysates were centrifuged for 15 minutes at 14,000 g, the protein concentration of supernatants was measured using BCA assay reagent (Pierce), and equivalent amounts of protein were separated on 7.5% SDS-PAGE gels and transferred to nitrocellulose. Target protein levels were assessed using antibodies to the β-subunit of the IGF1R or IR (Santa Cruz), phospho-Ser 473 Akt or total Akt (Cell Signalling, New England Biolabs) or β-tubulin (Sigma). Activated IGF1R was detected by immunoprecipitation for phosphotyrosine (P-Tyr-100, Cell Signaling Technology). Primary antibodies were detected with HRP-conjugated secondary antibodies (Dako), and ECL Plus (Amersham Pharmacia).

Results

(i) Down-Regulation of IGF1R in MDA-231 Cells.

The ASOs and RNAi duplexes were transfected into MDA-231 cells (human ER negative breast cancer) using Cytofectin or Oligofectamine, as described above. After 48 hr the cells were lysed, and IGF1R and IR levels were measured by immunoblotting. The intensity of the autoradiographic bands was quantified by densitometry, and IGF1R (or IR) levels were corrected for loading differences. The specific IGF1R or IR results are presented as % IGF1R (or IR) level of that in cells transfected with the same concentration of an appropriate control. This was a scrambled control oligonucleotide for ASOs, and an inverted RNA duplex for RNAi. The results are shown in Table 3 and FIG. 3.

TABLE 3
Effect of IGF1R-ASOs on IGF1R and IR
expression in MDA-231 cells

| Protein Level (% control) | |
|---|---|
| IGF1R | IR |

| ASO | 30 nM | 300 nM | 30 nM | 300 nM |
|---|---|---|---|---|
| 1 | 66 ± 23 | 38 ± 6 | 112 | 135 |
| 2 | 84 ± 11 | 47 ± 5 | nd | nd |
| 3 | 105 ± 18 | 104 ± 22 | nd | nd |
| 4 | 73 ± 2 | 49 ± 2 | 97 | 92 |
| 6 | nd | ˜150 | nd | 100 |
| TSS | 141 ± 10 | 64 ± 19 | 144 | 59 |

It is to be noted that whilst the ASOs identified as hybridising strongly with IGF1R mRNA but not IR mRNA on the basis of scanning array screening (ASOs 1 and 4) did not cause significant downregulation of IR, there was a detectable reduction in IR levels in cells treated with 300 nM TSS ASO. Thus, ASOs 1 and 4 exhibit greater selectivity for IGF1R mRNA versus IR mRNA. NB-ASO2 was not tested for IR downregulation.
(ii) Comparison of 18mers (eg ASO1/18, ASO2/18) Versus 20mers (ASO1/20, ASO2/20)

IGF1R downregulation in UC101 ovarian cancer cells was observed with ASO1 (18mer) and ASO2 (18mer), as summarised in Table 4.

TABLE 4
Effect of 18mer ASOs in IGF1R expression in UC101 cells

| | |
|---|---|
| ASO1/18mer | 45% of levels in cells treated with Scr1 |
| ASO2/18mer | 39% of levels in cells treated with Scr2 |
| TSS ASO | 33% of levels in cells treated with |
| | ScrTSS |
| ASO6 | 98% of levels in cells treated with Scr6 |

These effects are similar to relative IGF1R downregulation observed using ASO1 and ASO2 20mers. FIG. 9 illustrates the efficacy of ASO1, ASO2, ASO6 and TSS 18mer antisense oligonucleotides in causing IGF1R downregulation in UC101 ovarian cancer cells. Results are expressed as % IGF1R expression in cells transfected with a scrambled control sequence.
(iii) Comparison of the Effects of ASOs and RNAi

The effects of an ASO and RNAi duplex of equivalent sequence (ASO2 and R2) on IGF1R and IR expression were compared in MDA-231 human breast cancer cells and ME melanoma cells. The transfection protocol was as above, using either Cytofectin (C) or Oligofectamine (O; Gibco BRL). After 48 hr the cells were lysed and IGF1R and IR levels were determined by immunoblotting. Representative immunoblots are shown in FIG. 4.
a) MDA-231 breast cancer cells transfected with ASO 2 (or scrambled control, Scr) or RNAi (sequence R2 given above) or inverted RNA duplex control (InvRNA) at 20 or 200 nM.
b) MDA-231 cells were transfected with Oligofectamine and RNAi or Inverted control (InvRNA) at 0.1-10 nM.
c) ME melanoma cells were transfected with Oligofectamine and RNAi or Inv control duplex at 5-500 nM.
   Quantitative analysis of these results is incorporated into the graph of ASO effects in MDA-231 (FIG. 3).

It is clear that RNAi causes much more profound inhibition of IGF1R expression than occurs with ASOs. Note that the quantification methods may not be linear at very low protein levels, and it is possible that these analyses underestimate the true extent of IGF1R downregulation. For example the immunoblots (FIG. 4) show almost complete inhibition of IGF1R expression (even on these overnight exposures) in MDA-231 breast cancer and ME melanoma cells treated with 10-200 nM RNAi.

A second RNAi (R6 in FIG. 5 and Table 2) was synthesised corresponding to ASO 6, which hybridizes to IGF1R mRNA on the array with a relative intensity of 0.01. While this does cause downregulation of the IGF1R, the effects were significantly less potent than that of the RNAi corresponding to ASO2 (R2 in FIG. 5 and Table 2). RNAi R2 is more effective than RNAi R6 in the following cell lines:

| | |
|---|---|
| DU145 | prostate |
| A549 | NSCLC |
| UC101 | ovary |
| U20S | osteosarcoma |
| MCF7 | breast (ER positive) |
| MDA-231 | breast (ER negative) |
| ME | human melanoma |
| B16 | mouse melanoma |

This result indicates that secondary structure in the target transcript influences the efficacy of small synthetic RNAis, as it does for ASOs. Moreover, it indicates that intensity of hybridisation to the scanning array is predictive of the activity of RNAi reagents.

The inhibitory effect of RNAi R2 is partially, though not completely, blocked by the presence of a single base pair mutation (sequence Mut2 in FIG. 5 and Table 2), see FIG. 5, compare R2 with Mut2. The effect of R6 was less than the effect of the mutant duplex Mut2. This indicates that the efficacy of synthetic 21mer RNAi molecules is influenced by secondary structure in, and hence access to, the target region of the mRNA.
(iv) Comparison of RNAi Duplexes R2 and R6 in a Range of Human and Murine Cell Lines

Tumour cells were transfected with 10 nM duplexes and IGF1R levels were measured after 48 hr. After correction for loading differences IGF1R levels in cells transfected with RNAi were expressed as % of levels in cells transfected with equivalent inverted control duplex.

TABLE 5
Effect of R2 and R6 RNAi duplexes in human and murine cell lines

| Cell line | Cell type | R2 | R6 |
|---|---|---|---|
| DU145 | Human prostate cancer | 34 ± 13 | 74 ± 8 |
| A549 | Human non-small cell lung | 22 ± 2 | 102 ± 16 |
| ME | Human melanoma | 53 ± 3 | 104 ± 5 |
| B16 | Murine melanoma | 45 ± 4 | 72 ± 14 |

In some cell lines, e.g. human ME melanoma and A549 NSCLC, 10 nM R6 was essentially inactive. In all cases R2 caused more profound IGF1R downregulation than R6 (FIG. 5b). This suggests that the structural features dictating access are robust and conserved between different cell lines and species.
(v) Effect of RNAi Duplexes of Varying Length

Compared with the effects of R2, IGF1R downregulation is less profound after treatment with an 18mer duplex (sequence as R2 but lacking the 3 RNA bases at the 3′ end). In contrast to the results of Elbashir et al. (2001), a 24mer R2 duplex was observed to be as effective as R2 at 100 nM. At 0.5 and 5 nM the 24mer still caused detectable IGF1R down-regulation but was less effective than the 21mer R2. Results are illustrated in FIG. 6: Panel (a) is an immunoblot showing the effect of various siRNAs on IGF1R expression at 0.5 and 5 nM; panel (b) is a graphical illustration showing the effect of RNAis of varying length, results are presented as % IGF1R level of that in cells transfected with the same concentration of an Inv2 control RNAi. A 27mer duplex, representing a 6 bp 3′ extension of R2 (see Table 2) induced comparable IGF1R downregulation (see FIG. 6(a)). This is in contrast to duplex length requirements for RNAi in Drosophila (Elbashir, S. M. et al., EMBO J, Vol 20, 6877-6888, 2001) but consistent with recently reported characteristics of RNAi in mammalian cytoplasmic lysates (Martinez, J. et al., Cell, Vol 110, 563, 2002).

(vi) Testing of RNAi Duplexes to a Further Region of Intense Hybridisation between IGF1R mRNA and the IGF1R Scanning Array

A second pair of duplexes, R4 and R5, were based on the sequence around peak 4 of hybridization between IGF1R mRNA and the IGF1R ASO scanning array (FIG. 11). Duplexes R4 and R5 were designed to target 19mer sequences immediately downstream of AA motifs at bases 636 and 639 respectively of the IGF1R sequence (Ullrich, A. et al., EMBO J, Vol 5, 2503-2512, 1986). Data from the scanning array indicated that there was a ˜6.5 fold difference in hybridization intensity between the equivalent 19mer ASOs (FIG. 11B). Both duplexes induced IGF1R downregulation in human ovarian and prostate cancer cells, but R4 was significantly more potent (p<0.05) in both cell lines (FIGS. 11C, D). Thus a 3 bp shift had major effects on siRNA efficacy, parallelling differences in heteroduplex yield on the scanning array.

The R4 siRNA duplex has induced significant IGF1R downregulation (IGF1R levels 10-20%) compared with levels in cells treated with inverted control or scrambled sequence duplex, in the following cell lines:

| | |
|---|---|
| MDA-MB-231 | human estrogen receptor negative breast |
| | cancer |
| MCF-7 | human estrogen receptor positive breast |
| | cancer |
| T47D | human estrogen receptor positive breast |
| | cancer |
| A549 | human non-small cell lung cancer |
| HeLa | human cervical cancer |
| UC101 | human ovarian cancer |
| DU145 | human androgen-resistant prostate |
| | cancer |
| ME | human melanoma (and 7 further human |
| | melanoma cell lines: A2058, SKmel 2, |
| | SKmel 23, SKmel 29, SKmel 31, CHL-1, |
| | HMCB) |
| HEK293 | human kidney fibroblast |
| B16 | murine melanoma |

### EXAMPLE 6

### Differential Effects of RNAi Duplexes on IGF Signalling

MDA-MB-231 cells transfected with 100 nM RNA duplexes were serum-starved overnight and treated with 50 nM IGF-1 or diluent for 30 min. The cells were lysed and lysates were analysed by immunoblotting for IGF1R, phospho-Ser473-Akt and total Akt, and by phosphotyrosine immunoprecipitation and IGF1R immunoblotting to detect phosphorylated IGF1R β-subunit (see FIG. 7).

The relatively modest IGF1R downregulation induced by ASOs 1-5 or siRNA R6 was not sufficient to abolish IGF-I signaling to Akt (FIG. 7A and data not shown). In contrast transfection with R2 induced profound IGF1R gene silencing, such that we could not detect IGF-1-induced phosphorylation of the IGF1R β-subunit or of Akt. This is significant because it indicates that R2 not only caused more potent IGF1R downregulation but that this translated to a significant biological advantage; Akt phosphorylation is the major anti-apoptosis pathway downstream of the IGF1R (Baserga, et al., Biochem Biophys Acta, Vol 1332, F105-126, 1997).

The effect of transfection with 100 nM ASO4 or siRNA duplexes R4 and R5, equivalent to strongly and weakly hybridizing ASOs respectively, on colony forming efficiency and post-irradiation survival was also evaluated. The phosphorothioate ASO4 caused inhibition of overall survival and survival after 2Gy irradiation (SF2), but this effect was entirely non-specific, with similar results in cultures treated with the scrambled sequence oligonucleotide (Scr4, FIGS. 7B, C). Transfection with siRNAs R4 or R5 inhibited survival of MDA-MB-231 (p<0.001 for comparison of each siRNA with inverted control duplex), with a significantly greater effect in R4-treated cells (p<0.01 for comparison with R5; FIG. 7B). Only in cultures treated with the more potent siRNA duplex R4 was there significant inhibition of survival after 2Gy irradiation (SF2; p<0.01 for comparisons with Inv4 and R5; FIG. 7C). These results confirm that the complex secondary structure of target transcripts can influence siRNA efficacy, and that these differences are sufficient to confer major variation in biological efficacy of potential therapeutic significance. This in turn illustrates the utility of hybridisation to the scanning ASO array in the selection of siRNA reagents, since relative hybridisation to the array is found to be predictive of activity in RNA interference and biological efficacy.

### EXAMPLE 7

### Growth of Cells Transfected with RNAi Duplexes In Vivo

B16 cells were transfected with 200 nM RNA duplexes (RNA22=R2, 22Inv=Inv2) or oligofectamine alone (OF) for three consecutive days. On the 4th day cells were injected into the flanks of C57BL mice, using 2.5×10⁵ cells/mouse, using groups of 5 mice. Tumours were measured in 2 dimensions every 2-3 days. Tumour volumes were calculated as n(a×b²)/6 where a is the larger and b the smaller dimension.

B16 cells transfected with R2 showed reduced growth rate in vivo, (syngeneic C57BL mice) compared with cells transfected with inverted control RNA duplex (FIG. 8). This results indicates that an RNAi reagent selected on the basis of hybridisation to a scanning array is effective in vivo.

Growth of Tumours In Vivo:

Tumorigenicity testing was conducted using UC101 human ovarian cancer cells transfected with 200 nM siRNA. After 48 hr the cells were inoculated into the flanks of female athymic (nu/nu mice), using 5×10⁶ tumour cells mixed with 5×10⁶ irradiated NIH-3T3 cells per mouse. Tumours were measured and volumes calculated as described (Macaulay et al., Oncogene 20: 2029-40, 2001). All procedures were performed in accordance with the Home Office Scientific Procedures Act 1986, and with the approval of the Animal Ethics Committee of Cancer Research UK.

Results:

Mice inoculated with UC101 cells transfected with siRNAs R5 or R6 developed rapidly growing tumours necessitating sacrifice within 30 days. Two of 4 mice injected with R4-transfected tumour cells remained tumour-free >60 days. The remaining two developed tumours that initially grew more slowly (at 18 days mean tumour size smaller than tumours in animals inoculated with R5-transfected cells, p<0.01), although the growth rate thereafter parallelled that of the controls (not shown). Thus a single transfection with R4 abolished tumour formation in 2/4 animals, and in the remaining two caused significant tumour growth delay that persisted for ˜18 days (FIG. 15).

### EXAMPLE 8

### Further Data on Activity of the R4 Duplex

Example 6 shows that duplex R4 blocks clonogenic survival in monolayer assays (ie in context of anchorage-dependent growth; FIG. 7B) and enhanced tumour cell radiosensitivity (FIG. 7C). In addition, it is observed that transfection with R4 causes significant inhibition of anchorage-independent growth of A549 non-small cell lung cancer cells (FIG. 12) and enhanced tumour cell chemosensitivity. This has been shown in DU145 prostate cancer cells, where R4 enhanced etoposide-induced apoptosis, measured by annexin staining and by caspase 3/7 activation assay (FIG. 13). In MDA-MB-231 cells transfected with R4, sensitivity to etoposide was significantly enhanced as shown by clonogenic assay (FIG. 14).

### EXAMPLE 9

### Stability of siRNAs

Although single-stranded RNA is highly unstable in serum, the siRNA duplexes appear to be significantly more stable (FIG. 16), presumably by virtue of the fact that they are double-stranded, and possess DNA ends.

### EXAMPLE 10

### Activity OF siRNA TO TSS

An siRNA reagent targeting a region of the IGF1R transcript nearer to the translation start site (TSS) of the IGF1R mRNA was designed as described (Elbashir et al Nature 411: 494-498, 2001 and www.dharmacon.com) by locating a region of mixed sequence >75 bp downstream of the translation start site. The selected 19-mer sequence was immediately downstream of an AA doublet at bases 166-7 of the human IGF1R sequence. Thus it is a 21mer duplex homologous to nucleotides 166-186 of the IGF1R transcript. The sequence of siRNA TSS (SEQ ID NO:37) is as follows:

| | |
|---|---|
| Sense strand: | 5′- CGACUAUCAGCAGCUGAAGTT - 3′ |
| Antisense strand: | 3′- TTGCUGAUAGUCGUCGACUUC - 5′ |

The siRNA TSS has been shown to induce IGF1R gene silencing in MDA-MB-231 human breast carcinoma cells. It has also been extensively evaluated in cultured prostate cancer cells, where we had previously had only limited success in downregulating the IGF1R using antisense agents (Hellawell et al BJU International 91: 271-7, 2003).

The greater potency and reduced non-specific toxicity of the siRNA duplexes allowed investigation beyond the DU145 cells, to include PC3 (like DU145, androgen-resistant) and LNCaP (androgen-responsive). This enabled a comparison of the effect of IGF1R gene silencing in DU145 cells, where the major downstream signaling pathways are IGF-responsive, with the effect in LNCaP and PC3 cells which harbour PTEN mutations. PTEN is a phosphatase that antagonises the effect of PI3 kinase, resulting in constitutive activation of Akt and hence protection from apoptosis.

The siRNA TSS caused significant inhibition of IGF1R expression and clonogenic survival in all three cultured human prostate cancer cell lines (FIG. 17, 18). This may be because IGFs influence survival via multiple pathways, and/or because the Akt pathway may retain a degree of IGF responsiveness, even when basally activated by PTEN loss.

Furthermore, IGF1R gene silencing appeared to enhance prostate cancer radio- and chemo-sensitivity (FIG. 19). The alteration in sensitivity was calculated as change in IC₅₀, the concentration of drug/irradiation required to reduce the colony count to 50% of that in untreated cultures, as follows:

$$\frac{{IC}_{50} in cultures transfected with scrambled control duplex :}{{IC}_{50} in cultures transfected with TSS}$$

The result shown in FIG. 19 indicated that in PC3 androgen-independent prostate cancer cells, the siRNA TSS induced a −2 fold increase in sensitivity to mitoxantrone, a drug with limited anticancer activity in patients with prostate cancer. The ability to enhance the efficacy of mitoxantrone in the clinical setting could confer significant objective benefit, in addition to the symptomatic improvement that can be achieved currently. The Table below summarises the results of chemosensitivity testing in DU145 and PC3 cells.

| Agent | DU145 | PC3 |
|---|---|---|
| Irradiation | 1.9 | 1.9 |
| Mitoxantrone | 1.5 | 2.1 |
| Etoposide | 1.9 | 2.4 |

As with the effect on overall survival, the effect on chemosensitivity appeared to be at least as good in PTEN mutant PC3 cells as in DU145.

The siRNA TSS has also been used to target the IGF1R in human bladder cancer cells, where it induced profound inhibition of clonogenic survival, and enhancement of sensitivity to mitomycin-C, a cytotoxic drug with clinical anticancer activity in patients with bladder cancer (FIG. 20).

1. An siRNA reagent comprising a double-stranded RNA sequence of up to 30 consecutive nucleotides of the human IGF-1 receptor mRNA sequence.

2. An siRNA reagent according to claim 1 wherein the double-stranded RNA sequence consists of up to 30 consecutive nucleotides of the human IGF-1 receptor mRNA sequence between position 537 and position 685.

3. An siRNA reagent according to claim 1 wherein the double-stranded RNA is of identical sequence to an antisense oligonucleotide which hybridises to IGF1R mRNA with a relative intensity of at least 0.01 in a hybridisation buffer consisting of 1 M NaCl, 10 mM Tris-HCl pH 7.4, 1 mM EDTA, 0.01% SDS (w/v) at a temperature of 37° C.

4. An siRNA reagent according to claim 1 wherein the double-stranded RNA comprises between 18 and 30 consecutive nucleotides of the human IGF-1 receptor mRNA sequence.

5. An siRNA reagent according to claim 1 wherein the double-stranded RNA comprises between 20 and 27, preferably between 20 and 24, consecutive nucleotides of the human IGF-1 receptor mRNA sequence.

6. An siRNA reagent according to claim 1 which is selected from the following:

| | |
|---|---|
| 5′-GCCGAUGUGUGAGAAGACCTT-3′ | (SEQ ID NO:23) |
| 3′-TTCGGCUACACACUCUUCUGG-5′ | (SEQ ID NO:38) |
| 5′-GCCGAUGUGUGAGAAGTT-3′ | (SEQ ID NO:24) |
| 3′-TTCGGCUACACACUCUUC-5′ | (SEQ ID NO:39) |
| 5′-GCCGAUGUGUGAGAAGACCACCTT-3′ | (SEQ ID NO:25) |
| 3′-TTCGGCUACACACUCUUCUGGUGG-5′ | (SEQ ID NO:40) |
| 5′-CUACAUUGUGGGGAAUAAGTT-3′ | (SEQ ID NO:34) |
| 3′-TTGAUGUAACACCCCUUAUUC-5′ | (SEQ ID NO:49) |
| 5′-CAAUGAGUACAACUACCGCTT-3′ | (SEQ ID NO:30) |
| 3′-TTGUUACUCAUGUUGAUGGCG-5′ | (SEQ ID NO:45) |
| 5′-GCCCAUGUGUGAGAAGACCTT-3′ | (SEQ ID NO:29) |
| 3′-TTCGGGUACACACUCUUCUGG-5′ | (SEQ ID NO:44) |
| 5′-CUACAUUGUGGGGAACAAGTT-3′ | (SEQ ID NO:36) |
| 3′-TTGAUGUAACACCCCUUGUUC-5′ | (SEQ ID NO:51) |
| 5′-CGACUAUCAGCAGCUGAAGTT-3′ | (SEQ ID NO:37) |
| 3′-TTGCUGAUAGUCGUCGACUUC-5′ | (SEQ ID NO:52) |

7. An siRNA reagent comprising a double-stranded RNA which is of identical sequence to an antisense oligonucleotide which hybridises to IGF1R mRNA and which exhibits greater specificity of hybridisation to IGF1R mRNA than to insulin receptor mRNA in a hybridisation buffer consisting of 1 M NaCl, 10 mM Tris-HCl pH 7.4, 1 mM EDTA, 0.01% SDS (w/v) at a temperature of 37° C.

8. A pharmaceutical composition comprising an siRNA reagent according to any one of claims 1 to 7 and pharmaceutically acceptable carrier, diluent or excipient.

9. An siRNA reagent according to claim 1 for use as a medicament.

10. Use of an siRNA reagent according to claim 1 for the manufacture of a medicament for the treatment of cancer or psoriasis.

11. An expression vector encoding an siRNA reagent as defined in claim 1.

12. A pharmaceutical composition comprising an expression vector according to claim 11 and pharmaceutically acceptable carrier, diluent or excipient.

13. An antisense oligonucleotide comprising at least 15 nucleotides which hybridises to IGF1R mRNA with a relative intensity of greater than 0.28 in a hybridisation buffer consisting of 1 M NaCl, 10 mM Tris-HCl pH 7.4, 1 mM EDTA, 0.01% SDS (w/v) at a temperature of 37° C.

14. An antisense oligonucleotide according to claim 13 which is complementary to a region of the human IGF-1 receptor mRNA sequence between position 537 and position 685.

15. An antisense oligonucleotide according to claim 14 which is at least 18 nucleotides and is complementary to a region of the human IGF-1 receptor mRNA sequence between position 612 and position 661, preferably between position 612 and position 641 or between position 630 and position 661.

16. An antisense oligonucleotide according to claim 14 which has a sequence selected from:

| | |
|---|---|
| 5′-GGCTTCTCCTCCATGGTC, | (SEQ ID NO:1) |
| 5′-CGGCTTCTCCTCCATGGTCC, | (SEQ ID NO:2) |
| 5′-GGTCTTCTCACACATCGG, | (SEQ ID NO:6) |
| 5′-TGGTCTTCTCACACATCGGC, | (SEQ ID NO:7) |
| or | |
| 5′-GCGGTAGTTGTACTCATTGT | (SEQ ID NO:13) |

17. An antisense oligonucleotide comprising at least 15 nucleotides which hybridises to IGF1R mRNA and which exhibits greater specificity of hybridisation to IGF1R mRNA than to insulin receptor mRNA in a hybridisation buffer consisting of 1 M NaCl, 10 mM Tris-HCl pH 7.4, 1 mM EDTA, 0.01% SDS (w/v) at a temperature of 37° C.

18. A pharmaceutical composition comprising an antisense oligonucleotide according to claim 13 and pharmaceutically acceptable carrier, diluent or excipient.

19. An antisense oligonucleotide according to claim 13 for use as a medicament.

20. Use of an antisense oligonucleotide according to claim 13 for the manufacture of a medicament for the treatment of cancer or psoriasis.

21. An expression vector encoding an antisense oligonucleotide as defined in claim 13.

22. A pharmaceutical composition comprising an expression vector according to claim 21 and pharmaceutically acceptable carrier, diluent or excipient.

23. A method of preparing a double-stranded RNA for use in gene silencing of a target gene by RNA interference, which method comprises:
(a) preparing a scanning array of antisense oligonucleotides spanning a region of a transcript of the human IGF1R gene;
(b) hybridising to the array labelled transcripts of the IGF1R gene;
(c) identifying an oligonucleotide within the array which hybridizes with the labelled transcripts; and
(d) preparing an siRNA reagent comprising a double-stranded RNA of identical sequence to the oligonucleotide identified in step (c).

24. A method of preparing an expression vector capable of expressing an siRNA reagent for use in gene silencing of IGF1R by RNA interference, which method comprises:
(a) preparing a scanning array of antisense oligonucleotides spanning a region of a transcript of the human IGF1R gene;
(b) hybridising to the array labelled transcripts of the human IGF1R gene;
(c) identifying an oligonucleotide within the array which hybridizes with the labelled transcripts; and
(d) preparing an expression vector capable of expressing an siRNA comprising a double-stranded RNA of identical sequence to the oligonucleotide identified in step (c).

25. Method according to claim 23 wherein the scanning array comprises an array of antisense oligonucleotides complementary to the region from position 537 to position 685 in the human IGF1R cDNA sequence.

26. Method according to claim 23 wherein hybridisation carried out in a hybridisation buffer consisting of 1 M NaCl, 10 mM Tris-HCl pH 7.4, 1 mM EDTA, 0.01% SDS (w/v) at a temperature of 37° C., and step (c) comprises identifying oligonucleotides which hybridise with a relative intensity greater than 0.01.

27. A method of preparing a pharmaceutical composition comprising an siRNA reagent capable of mediating gene silencing of the human IGF1R gene by RNA interference, which method comprises:
preparing an siRNA reagent for use in gene silencing of a target gene by RNA interference according to the method of claim 23 and formulating the siRNA reagent into a pharmaceutical composition comprising the siRNA reagent and one or more diluents, exicipients or carriers.

28. A method of preparing a pharmaceutical composition comprising an expression vector capable of expressing an siRNA for use in gene silencing of IGF1R by RNA interference, which method comprises:
preparing an expression vector capable of expressing an siRNA for use in gene silencing of IGF1R by RNA interference according to the method of claim 24 and formulating the expression vector into a pharmaceutical composition comprising the expression vector and one or more diluents, exicipients or carriers.

29. A scanning array of antisense oligonucleotides of complementary to the region from position 537 to position 685 in the human IGF-1 receptor cDNA sequence.

30. An array according to claim 29 wherein the oligonucleotides are from 1 to 18 or 1 to 20 nucleotides in length.


SEQUENCE LISTING (71 sequences)

SEQ ID NO: 1 - DNA, 18 bp, Artificial
  Antisense Oligonucleotide
ggcttctcctccatggtc

SEQ ID NO: 2 - DNA, 20 bp, Artificial
  Antisense Oligonucleotide
cggcttctcctccatggtcc

SEQ ID NO: 3 - DNA, 20 bp, Artificial
  Antisense Oligonucleotide
gggcttctcctccaatgtcc

SEQ ID NO: 4 - DNA, 20 bp, Artificial
  Antisense Oligonucleotide
tcttccgcgacttgctccgc

SEQ ID NO: 5 - DNA, 20 bp, Artificial
  Antisense Oligonucleotide
ctgttcaccgtgcaccctgt

SEQ ID NO: 6 - DNA, 18 bp, Artificial
  Antisense Oligonucleotide
ggtcttctcacacatcgg

SEQ ID NO: 7 - DNA, 20 bp, Artificial
  Antisense Oligonucleotide
tggtcttctcacacatcggc

SEQ ID NO: 8 - DNA, 20 bp, Artificial
  Antisense Oligonucleotide
tggtcttctcacacatgggc

SEQ ID NO: 9 - DNA, 20 bp, Artificial
  Antisense Oligonucleotide
cctgtacgcgttgatctcca

SEQ ID NO: 10 - DNA, 20 bp, Artificial
  Antisense Oligonucleotide
agtcgcctagtcgagtcctt

SEQ ID NO: 11 - DNA, 20 bp, Artificial
  Antisense Oligonucleotide
attgttgatggtggtcttct

SEQ ID NO: 12 - DNA, 20 bp, Artificial
  Antisense Oligonucleotide
tcatgcttgttgatgtggtt

SEQ ID NO: 13 - DNA, 20 bp, Artificial
  Antisense Oligonucleotide
gcggtagttgtactcattgt

SEQ ID NO: 14 - DNA, 20 bp, Artificial
  Antisense Oligonucleotide
tgtcgttcgagttgatcgta

SEQ ID NO: 15 - DNA, 20 bp, Artificial
  Antisense Oligonucleotide
gtggtccagcagcggtagtt

SEQ ID NO: 16 - DNA, 20 bp, Artificial
  Antisense Oligonucleotide
gacgttagcgtgcgatgtgc

SEQ ID NO: 17 - DNA, 18 bp, Artificial
  Antisense Oligonucleotide
cttattccccacaatgta

SEQ ID NO: 18 - DNA, 18 bp, Artificial
  Antisense Oligonucleotide
cttgttccccacaatgta

SEQ ID NO: 19 - DNA, 18 bp, Artificial
  Antisense Oligonucleotide
catagcacattcttctca

SEQ ID NO: 20 - DNA, 18 bp, Artificial
  Antisense Oligonucleotide
accgttcatcagttcact

SEQ ID NO: 21 - DNA, 18 bp, Artificial
  Antisense Oligonucleotide
tcctccggagccagactt

SEQ ID NO: 22 - DNA, 18 bp, Artificial
  Antisense Oligonucleotide
cagctactcgcatgctgc

SEQ ID NO: 23 - RNA, 21 nt, Artificial
  siRNA reagent
gccgaugugugagaagaccnn

SEQ ID NO: 24 - RNA, 18 nt, Artificial
  siRNA reagent
gccgaugugugagaagnn

SEQ ID NO: 25 - RNA, 24 nt, Artificial
  siRNA reagent
gccgaugugugagaagaccaccnn

SEQ ID NO: 26 - RNA, 27 nt, Artificial
  siRNA reagent
gccgaugugugagaagaccaccaucnn

SEQ ID NO: 27 - RNA, 21 nt, Artificial
  siRNA reagent
ccagaagaguguguagccgnn

SEQ ID NO: 28 - RNA, 21 nt, Artificial
  wherein “n” represents “t” thymine
gccgaugugugugaagaccnn

SEQ ID NO: 29 - RNA, 21 nt, Artificial
  siRNA reagent
gcccaugugugagaagaccnn

SEQ ID NO: 30 - RNA, 21 nt, Artificial
  siRNA reagents
caaugaguacaacuaccgcnn

SEQ ID NO: 31 - RNA, 21 nt, Artificial
  siRNA reagent
cgccaucaacaugaguaacnn

SEQ ID NO: 32 - RNA, 21 nt, Artificial
  siRNA reagent
ugaguacaacuaccgcugcnn

SEQ ID NO: 33 - RNA, 21 nt, Artificial
  siRNA reagent
cgucgccaucaacaugagunn

SEQ ID NO: 34 - RNA, 21 nt, Artificial
  siRNA reagent
cuacauuguggggaauaagnn

SEQ ID NO: 35 - RNA, 21 nt, Artificial
  siRNA reagent
gaauaagggguguuacaucnn

SEQ ID NO: 36 - RNA, 21 nt, Artificial
  siRNA reagent
cuacauuguggggaacaagnn

SEQ ID NO: 37 - RNA, 21 nt, Artificial
  siRNA reagent
cgacuaucagcagcugaagnn

SEQ ID NO: 38 - RNA, 21 nt, Artificial
  siRNA reagent
ggucuucucacacaucggcnn

SEQ ID NO: 39 - RNA, 18 nt, Artificial
  siRNA reagent
cuucucacacaucggcnn

SEQ ID NO: 40 - RNA, 24 nt, Artificial
  siRNA reagent
gguggucuucucacacaucggcnn

SEQ ID NO: 41 - RNA, 27 nt, Artificial
  siRNA reagent
gaugguggucuucucacacaucggcnn

SEQ ID NO: 42 - RNA, 21 nt, Artificial
  siRNA reagent
cggcuacacacucuucuggnn

SEQ ID NO: 43 - RNA, 21 nt, Artificial
  siRNA reagent
ggucuucacacacaucggcnn

SEQ ID NO: 44 - RNA, 21 nt, Artificial
  siRNA reagent
ggucuucucacacaugggcnn

SEQ ID NO: 45 - RNA, 21 nt, Artificial
  siRNA reagent
gcgguaguuguacucauugnn

SEQ ID NO: 46 - RNA, 21 nt, Artificial
  siRNA reagent
guuacucauguugauggcgnn

SEQ ID NO: 47 - RNA, 21 nt, Artificial
  siRNA reagent
gcagcgguaguuguacucann

SEQ ID NO: 48 - RNA, 21 nt, Artificial
  siRNA reagent
acucauguugauggcgacgnn

SEQ ID NO: 49 - RNA, 21 nt, Artificial
  siRNA reagent
cuuauuccccacaauguagnn

SEQ ID NO: 50 - RNA, 21 nt, Artificial
  siRNA reagent
gauguaacaccccuuauucnn

SEQ ID NO: 51 - RNA, 21 nt, Artificial
  siRNA reagent
cuuguuccccacaauguagnn

SEQ ID NO: 52 - RNA, 21 nt, Artificial
  siRNA reagent
cuucagcugcugauagucgnn

SEQ ID NO: 53 - DNA, 15 bp, Artificial
  Oligonucleotide
attgtggggaataag

SEQ ID NO: 54 - DNA, 15 bp, Artificial
  Oligonucleotide
catggaggagaagcc

SEQ ID NO: 55 - DNA, 15 bp, Artificial
  Oligonucleotide
atgtgtgagaagacc

SEQ ID NO: 56 - DNA, 15 bp, Artificial
  Oligonucleotide
agaccaccatcaaca

SEQ ID NO: 57 - DNA, 15 bp, Artificial
  Oligonucleotide
gagtacaactaccgc

SEQ ID NO: 58 - DNA, 15 bp, Artificial
  Oligonucleotide
ccgctgctggaccac

SEQ ID NO: 59 - DNA, 150 bp, Homo sapiens
tgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaa

SEQ ID NO: 60 - DNA, 18 bp, Artificial
  Oligonucleotide
tacattgtggggaataag

SEQ ID NO: 61 - DNA, 17 bp, Artificial
  Oligonucleotide
gaccatggaggacaacc

SEQ ID NO: 62 - DNA, 18 bp, Artificial
  Oligonucleotide
ccgatgtgtgagaagacc

SEQ ID NO: 63 - DNA, 18 bp, Artificial
  Oligonucleotide
agaagaccaccatcaaca

SEQ ID NO: 64 - DNA, 18 bp, Artificial
  Oligonucleotide
aatgagtacaactaccgc

SEQ ID NO: 65 - DNA, 18 bp, Artificial
  Oligonucleotide
ctaccgctgctggaccac

SEQ ID NO: 66 - DNA, 150 bp, Artificial; identical to SEQ ID NO: 59
  Oligonucleotide

SEQ ID NO: 67 - DNA, 150 bp, Homo sapiens; identical to SEQ ID NO: 59

SEQ ID NO: 68 - DNA, 1001 bp, Homo sapiens
ttttttttttttttgagaaagggaatttcatcccaaataaaaggaatgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggacaattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcat

SEQ ID NO: 69 - DNA, 19 bp, Artificial
  Oligonucleotide
caatgagtacaactaccgc

SEQ ID NO: 70 - DNA, 19 bp, Artificial
  Oligonucleotide
tgagtacaactaccgctgc

SEQ ID NO: 71 - DNA, 15 bp, Artificial
  Antisense Oligonucleotide
gtgcagcagcggtag